(19) United States
(12) Patent Application Publication
(10) Pub. No.: US 2026/0079109 A1
(43) Pub. Date: Mar. 19, 2026

(54) FLUORESCENT SENSORS FOR MARIJUANA BREATH TEST

(71) Applicants: University of Utah Research Foundation, Salt Lake City, UT (US); Gentex Corporation, Zeeland, MI (US)

(72) Inventors: Ling ZANG, Salt Lake City, UT (US); Yanke CHE, Salt Lake City, UT (US); Jiangfan SHI, Salt Lake City, UT (US)

(21) Appl. No.: 18/832,330

(22) PCT Filed: Mar. 27, 2024
(86) PCT No.: PCT/US2024/021740

§ 371 Date: Jul. 23, 2024

Related U.S. Application Data
(60) Provisional application No. 63/496,314, filed on Apr. 14, 2023.
(60) Provisional application No. 63/589,702, filed on Oct. 12, 2023.

(51) Int. Cl. G01N 21/64; C07D 417/10; G01N 33/94

(52) U.S. Cl. CPC G01N 21/64; C07D 417/10; G01N 33/948

(57) ABSTRACT

A fluorescent sensor compound for detecting marijuana can include an oligomer having a general structure of Y-[D]ₙ-A-Z. In this structure, D is an electron donor group according to Formula (I), and A is an electron acceptor group according to Formula. (II). Additionally, Y is R₃, R₃-A, or R₃-D-A; Z is R₃ or [D]ₘ-R₃; n is an integer from 1 to 3; m is an integer from 1 to 2; R₁ is an alkyl chain and R₁ can optionally include different alkyl chains in different respective D groups; R₃ is an alkoxyphenyl end group. A fluorescent sensor for detecting marijuana (100) can include the fluorescent sensor compound formed as a. film (110) and a. fluorescence detector (150) oriented to detect a fluorescence response from the fluorescent sensor compound.

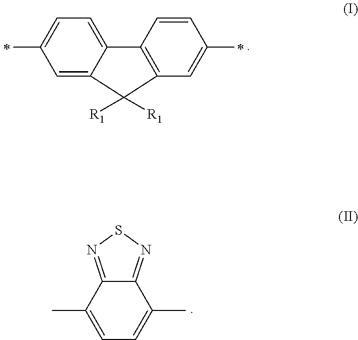

### CROSS REFERENCE TO RELATED APPLICATIONS

This application claims priority to U.S. Provisional Application No. 63/496,314, filed Apr. 14, 2023 and U.S. Provisional Application No. 63/589,702, filed Oct. 12, 2023, which are incorporated herein by reference.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION BY REFERENCE STATEMENT

Not applicable.

### BACKGROUND

As recreational use of marijuana becomes legalized nationwide, quick and reliable monitoring of the drug use becomes more useful to promote both personal and public safety. Use of marijuana may impair the sense and judgment of a user, causing loss of focus, and thereby seriously influencing activities such as driving and working. The primary psychoactive component of marijuana is tetrahydrocannabinol (THC), which stimulates the brain response to pleasure. The other major component present in marijuana is cannabidiol (CBD), which is not psychoactive, rather better recognized for its medical applications. While both THC and CBD can be considered as molecular markers of marijuana, THC has been the dominant target to be monitored, especially in roadside tests for driving under influence of drug (DUID), mainly due to its psychological activity.

### SUMMARY

This disclosure describes compounds that can be used to detect marijuana. Specifically, the compounds include fluorescent molecules that exhibit a change in fluorescence response when exposed to marijuana markers such as THC and CBD. In some examples, a fluorescent sensor compound for detecting marijuana can include an oligomer having a general formula structure of Y-[D]ₙ-A-Z wherein D is an electron donor group according to Formula (I):

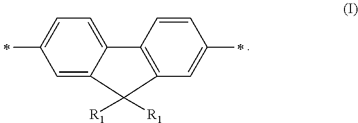

The A group can be an electron acceptor group according to Formula (11):

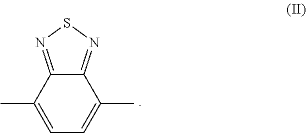

In these structures, Y can be R₃, R₃-A, or R₃-D-A; Z can be R₃ or [D]ₘ-R₃; i can be an integer from 1 to 3; m can be an integer from 1 to 2; R₁ can be an alkyl chain and R₁ can optionally include different alkyl chains in different respective D groups; R₃ can be an alkoxyphenyl end group.

Sensor devices are also described. The sensors can include the fluorescent sensor compound as described above. The sensors can also include a fluorescence detector oriented to detect a fluorescence response from the fluorescent sensor compound.

The present disclosure also describes methods of detecting a marijuana breath marker. An example method can include exposing a fluorescent sensor compound as described above to a breath sample, correlating a fluorescence response of the fluorescent sensor compound to a target threshold, and outputting an indicator corresponding to the presence or absence of the marijuana breath marker.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a schematic diagram of an example fluorescent sensor for detecting marijuana in accordance with examples of the present disclosure.

FIG. 2 is a schematic diagram of another example fluorescent sensor for detecting marijuana in accordance with examples of the present disclosure.

FIG. 3 is a flowchart illustrating an example method of detecting a marijuana breath marker in accordance with examples of the present disclosure.

FIG. 4 is a reaction scheme showing a synthesis route for synthesizing an example fluorescent sensor compound, TFT-C6, in accordance with examples of the present disclosure.

FIG. 5 is an NMR spectra of TFT-C6 as in the example of FIG. 4.

FIG. 6 is an MS spectra of TFT-C6 as in the example of FIG. 4.

FIG. 7 is an NMR spectra of another example fluorescent sensor compound, TFT-C4, in accordance with examples of the present disclosure.

FIG. 8A shows fluorescence quenching response to THC vapor at different temperatures in accordance with one example.

FIG. 8B shows fluorescence response to CBD vapor at different temperatures in accordance with one example.

FIGS. 9A and 9B show fitting curves and equation of the fluorescence quenching response obtained for (a) THC and (b) CBD at different vapor pressures (temperatures) in accordance with one example.

FIGS. 10A through 10G shows the fluorescence responses to the common potential interferents: (a) acetone (b) ethanol (c) isopropanol (d) water (relative humidity) (e) hexane (f) heptane (g) octane in accordance with one example.

FIG. 11A-11D show the fluorescence responses of (A-B) TFT-C6 and (C-D) TFT-C4 thin films towards 1, 2, 5, 10, and 20 ng of THC in accordance with one example.

FIGS. 12A and 12B show the linear fitting curves for LOD calculation of fluorescence responses of (a) TFT-C6 and (b) TFT-C4 towards vaporized THC in accordance with one example.

FIG. 13A shows fluorescent response of the compound of Structure XVIII towards 0.01 ng, 0.6 ng, 1 ng, and 1.5 ng of THC in accordance with one example.

FIG. 13B shows the linear fitting curve for LOD calculation of the fluorescent responses in FIG. 13A.

FIG. 14A is a graph of a detector response of 1 micro-liter saliva deposited on the PTFE testing strip in accordance with another example.

FIG. 14B is a graph of a detector response of 1 micro-liter saliva co-deposited with varying amounts of THC (10, 20, 100 pg) on the PTFE testing strip in accordance with another example.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a sensor” includes reference to one or more of such devices and reference to “exposing” refers to one or more of such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Fluorescent Sensor Compounds

The present disclosure describes fluorescent sensor compounds that can be used to detect marijuana. In particular, these compounds can exhibit a fluorescence response when exposed to certain marijuana markers, including THC and CBD Both of these markers can be present in the breath of a marijuana user at elevated levels for a time period after using marijuana. Therefore, the fluorescent sensor compounds can be exposed to the breath of a subject to determine the presence of THC or CBD in the breath. The magnitude of fluorescence response of these compounds can also be dependent on the concentration of THC or CBD in the breath. The concentration of THC and CBD in the breath can, in turn, be related to the length of time since the user has taken marijuana and the level of impairment of the user. Thus, the fluorescent sensor compounds described herein can provide information about the level of impairment of a user from taking marijuana.

The THC level in a user corresponds to the psychological effects experienced by the user. The THC level usually peaks in about 30 minutes after marijuana use, and may diminish in 1-3 hours. This 3-hour window can be considered as the peak impairment window that is aligned with DUID. After the peak time, THC can remain in the body as it is stored in fat cells for a much longer time. Indeed, THC remains detectable in saliva for up to 24 hours, in blood for 3 weeks, in urine for 1 month, and in hair for 3 months. Although testing of these samples may reveal that someone has recently used the drug, it cannot determine whether the subject is currently under the influence. So, detection technologies and methods based on testing of fluidic samples are not suited for roadside DUID testing or other scenarios where the goal is to determine whether the subject is under drug influence within the 3-hour impairment window.

The current testing of fluidic sample includes two categories of technology or method. One is the typical bench-top instrumentation such as those based on gas or liquid chromatography coupled with mass spectrometry, namely GC-MS or LC-MS. These instruments are superb in identification and quantification of THC, especially at low concentration level, but they are bulky, heavy, complicated, time consuming and expensive in operation, thus not suited for quick onsite or mobile monitoring of drug. The other category of detection technology mostly relies on chemical sensors including those based on Duquenois-Levine reagent, azo dyes (Fast Blue B/BB), or Beam's CBD test. The Duquenois-Levine reagent works well for detection of THC, but has false positive with 4-hexylresorcinol. The Beam's CBD test works well for CBD, but not THC. Nonetheless, the results of the fluidic sample testing can only provide law enforcement information about whether a subject has ever used the drug. Currently, to determine if a driver is under the influence of marijuana, police officers rely on field sobriety tests. Sometimes, help of a drug recognition expert (DRE) may also be utilized.

In contrast, a breathalyzer-type marijuana test would be more useful for law enforcement and other situations where a fast, mobile test is desired. The levels of THC and CBD in the breath of a marijuana user can be very low, at trace levels, even within the 3-hour window after using marijuana. Therefore, it would be useful for a test to be capable of measuring THC and CBD at trace levels. It would also be useful for the test to operate with minimal interference from moisture or other common species present in the breath or in local environments. Some research in this area has involved detection using high-field asymmetric waveform ion mobility spectrometry (FAIMS). As occurred to many other IMS based detectors, selectivity or elimination of interference of other species (e.g., mint) remains a challenge. Other sensors have been attempted utilizing an array of optical sensors (e.g., thin-layer chromatography). Detection sensitivity remains a challenge, with these sensors. Additional attempts have made to create sensors that include carbon nanotubes for gas phase detection of THC. However, the intrinsic poor selectivity of carbon nanotubes may bring challenges to discriminate THC from many other species present in the breath and in the local environment, for which false positive or overestimation of THC presence could be a serious concern in practical applications. In brief, it is difficult to provide a sensor for detection of THC in breath because of limitations in detection sensitivity and/or selectivity.

The fluorescent sensor compounds herein can be capable of detecting THC and CBD with high sensitivity and selectivity. The compounds can be used to make sensors that can help law enforcement determine instantly if a subject is under the influence of marijuana. The sensors described herein can detect THC, CBD, or both within the 3-hour impairment window, correlating the level of THC or CBD directly to the influence of the drug, or DUID. The detection can be both sensitive and selective in order to enable quantification of THC and CBD at trace level in breath, and with minimal interference potentially caused by moisture and other species existing in the breath and local environment. In some examples, the average concentration of THC present in exhaled breath can be 0.9 ng/L, 0.14 ng/L, or 0.05 ng/L (corresponding to 0.07 ppb, 0.01 ppb, and 0.004 ppb) as measured in samples collected immediately after, 1 hour after, or 3 hours after smoking marijuana, respectively. Average tidal volume of human breath is 500-600 mL, while an expiratory reserve volume (ERV) is about 1100 mL for males and 800 mL for females. The concentration of THC detected in the breath have been found to correlate with THC levels found in the blood during the 3-hour period after marijuana use. Therefore, the sensors can provide strong evidence for law enforcement to show that a subject has used marijuana within the 3-hour window. The sensors can also be simple and quick to use, which is highly desirable for rapid onsite screening or monitoring of drug use. Additionally, the sensors can utilize noninvasive sampling, e.g., from breath, rather than blood or urine. Compared to the current testing methods based on fluidic samples, breath analysis of THC would be an alternative approach for DUID testing for marijuana. In some examples, the sensors described herein can be in the form of a handheld breathalyzer-like device, which can be used for DUID testing for marijuana as simply as the current breathalyzer used for roadside DUI testing for alcohol.

A unique class of fluorescent sensor compounds is described below. These compounds can selectively detect THC, as well as CBD, in gas phase with no or controllable interference from the common species existing in breath or environment, such as moisture (humidity), alcohol, acetone (as used in nail polish liquid), gasoline, cigarette smoke, perfumes, sun creams, among many others. Under elevated temperature, such as above 60° C., the limit of detection (LOD) as tested for THC and CBD can reach 0.22 ppb for THC and 0.04 ppb for CBD or 5 pg for THC upon complete vaporization under high temperature e.g., 170° C. THC and CBD are involatile compounds, with saturated vapor concentration under room temperature of only 0.25 and 0.03 ppb, respectively. The melting point of THC and CBD are 64° C. and 68° C., respectively. Heating the sample above 60° C. can significantly increase the detection efficiency by maintaining more of THC in the gas phase. In some examples, such heating can be provided by adding a heater in front of the detector, which can quickly heat a sample and vaporize THC and CBD before the markers reach the detector. LODs of sensors described herein can be sufficient for direct detection of THC and CBD in a breath sample within the impairment window after cannabis use. In further examples, a preconcentration device can be associated with breath sample collection. For example, a preconcentration membrane similar to membranes used for solid phase extraction (SPE) can be incorporated into a mouthpiece used for breath sample collection. Typical SPE membranes can concentrate chemical analytes by a factor of 1,000 or higher.

The fluorescent sensor compounds described herein can include rigid linear oligomers containing fluorene and benzothiadiazole groups. These oligomers are electron donor-acceptor molecules in a conjugation structure, with fluorene acting as donor and benzothiadiazole as acceptor. These molecules are strongly fluorescent in both solution and solid state. For example, these compounds were deposited in a thin film inside a quartz or glass tube and found to be fluorescent in this form. Upon interaction with THC or CBD, the fluorescence emission of the film will be effectively quenched due to the higher electron density on the main chain when the molecules interact with the phenol groups of the THC/CBD by π-π interaction and increased dipole-dipole interactions. The extent of the emission quenching can be measured and correlated to the gas phase concentration of THC. A calibration curve can be established, and then used to determine gas phase concentration of THC based on measured emission quenching.

An example sensor with a film of the fluorescent sensor compound was made and tested against potential interferents. Interestingly, moisture and many common chemicals such as alcohol, acetone, gasolines cause opposite sensor response compared to THC and CBD, i.e., increase in emission intensity, likely due to the non-specific swelling mechanism, meaning that the temporary reduction of the intermolecular dipole-dipole interactions enhances the emission. Nonetheless, the responses obtained for these interferents were minimal. For example, when tested under 54% relative humidity (equivalent to water vapor concentration of 12,455 ppm) the sensor response observed in this example was only +0.044%, compared to the −0.27% response obtained for THC tested under only 39.6 ppb (at 75° C.), which represents a concentration level about 5 orders of magnitude lower than that of moisture. When exposed to 72.5 ppm ethanol, a common substance present in breath after drinking, the sensor generated only +0.019% response in this example, whereas a much larger response (though in opposite direction), −0.27%, was obtained for THC, though tested under a much lower concentration, 39.6 ppb (at 75° C.). Besides, the fluorescence responses of interferents are not only in opposite direction, but also reversible and quick (i.e., recovered in a short period of time), which can make the irreversible, relatively slower fluorescence quenching (decrease) responses towards THC and CBD easy to be distinguished in the practical tests. The quick, reversable response of the interferents would not affect the quantification measurement of THC or CBD. Detailed results of the sensor testing are presented below.

Compared to the current chemical sensors and analytical instrumentation, like chromatography coupled with mass spectrometry, this invention offers the following features can provide onsite detection of marijuana use. The sensors can have high sensitivity, with LOD down to low ppb for gas phase or low pg for surface detection. This makes it possible to develop the sensor into a breathalyzer-like detector to serve law enforcement and other customers. By coupling with a solid phase extraction component, such as an extraction membrane in the mouthpiece, THC and CBD can be effectively concentrated on the extraction membrane, which would allow for detection of THC and CBD directly from the surface, and further increase the detection capability by lowering the LOD of breath analysis by two or more orders of magnitude. The sensors can also have high selectivity, minimizing false positives in detection, especially against the common interferents present in breath and the local environment. The sensors can be easy to use, low cost, stable, and suited for quick onsite detection. Rapid breath testing can provide instant answers to law enforcement personnel in the case of roadside DUID screening regarding whether drug use occurred within the 3-hour impairment window.

Turning now to the structure of the fluorescent sensor compounds, in some examples a fluorescent sensor compound can include an oligomer having a general structure of

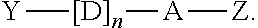

In this structure, D is an electron donor group according to Formula (I):

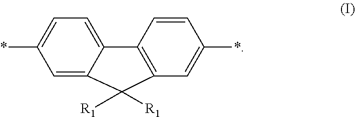

The structure also includes a group A, which is an electron acceptor group according to Formula (II):

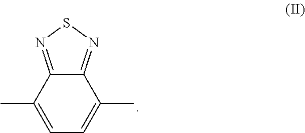

In the formulas for functional groups described herein, such as D and A above, a solid line terminating in a dot represents a bonding location where the functional group can bond to another atom or another functional group. The D and A groups each have two of these bonding locations, which means that each D and A group can bond with two other atoms or functional groups. The fluorescent sensor compounds described herein can include a linear oligomer that includes one ore more of each of the D and A groups. In Formula (I), R₁ is an alkyl chain. If the compound includes multiple D groups, then the R₁ groups may be different in different respective D groups. In Formula (II), the A is a benzothiadiazole group. Additionally, in the general structure Y-[D]ₙ-A-Z, Y can be R₃, R₃-A, or R₃-D-A. The Z group can be R₃ or [D]ₘ-R₃. In the Y and Z groups, R₃ can be an alkoxyphenyl end group. The subscripts in and m can be integers indicating a number of D groups bonded together linearly, where n is an integer from 1 to 3 and m is an integer from 1 to 2. In various examples, the fluorescent sensor compound can include a total number of A groups from 1 to 2, and a total number of D groups from 1 to 6. In certain examples, the fluorescent sensor compound can include a total number of A groups from 1 to 2 and a total number of D groups from 1 to 5.

Consistent with the above general formula, several more specific oligomer structures are shown in Formulas (III) through (VI). In some examples, the fluorescent sensor compound can include an oligomer according to one of these formulas.

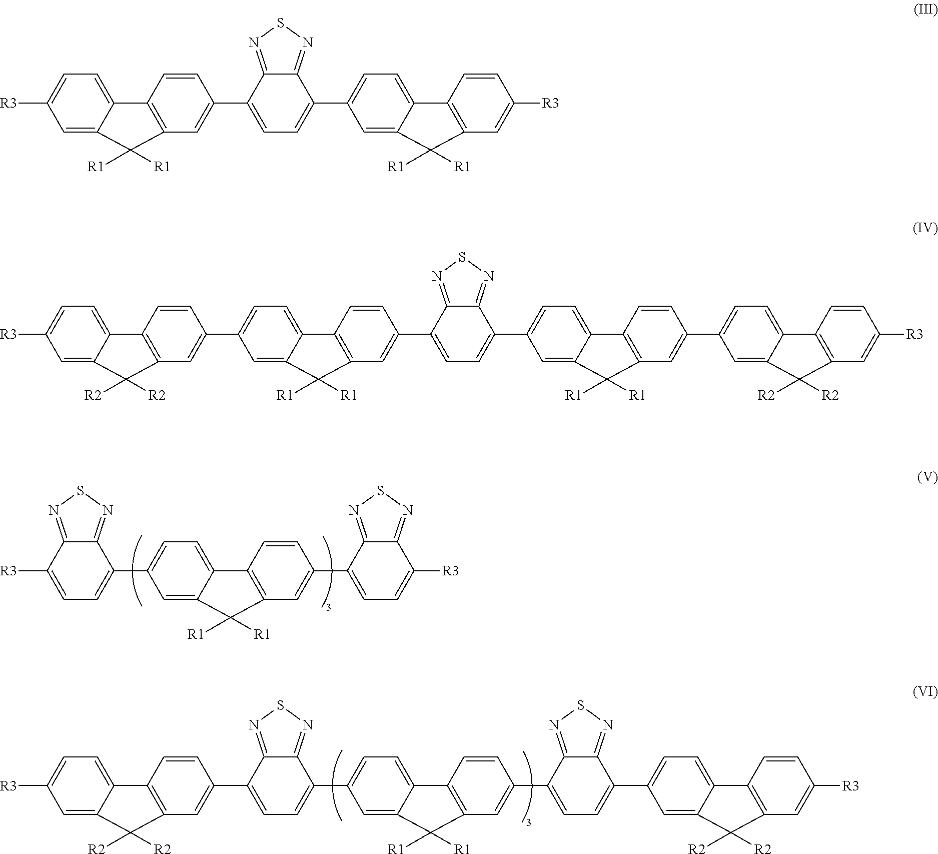

In these formulas, R₁, R₃, and X can be defined in the same way as in Formulas (I) and (II) above. The R₂ group can be an alkyl group similar to R₁. The use of both R₁ and R₂ can indicate that the R₁ group is one particular alkyl group and R₂ is a different alkyl group. However, in some examples, R₁ and R₂ can be the same alkyl group.

In certain examples, R₁ and R₂ can be independently selected from the group consisting of —CH₃, —C₂H₅, —C₃H₇, —C₄H₉, —C₅H₁₁, C₆H₁₃, C₇H₁₅, and C₈H₁₇. In some cases, the R₁ and R₂ groups can be linear alkyl groups. In other cases, the R₁ and R₂ can be independently linear alkyl chains having from 1 to 8 carbon atoms.

As mentioned above, the R₃ group can be an alkoxyphenyl end group. The alkoxyphenyl end group can include a phenyl group and an alkoxy group. The phenyl group can be directly bonded to the remainder of the oligomer, and the alkoxy group can be bonded to the phenyl group. In some examples, the alkoxy group can be bonded to the phenyl group at the para-position, i.e., opposite from the bond to the remainder of the oligomer. The alkoxy group can include a variety of linear alkoxy groups or branched alkoxy groups. In some examples, the alkoxy group can include an oxygen atom and from 1 to 10 carbon atoms. In further examples, the alkoxy group can include an oxygen atom and from 1 to 6 carbon atoms or from 1 to 5 carbon atoms. Specific examples of alkoxy groups are shown in Formulas VII through XVI below:

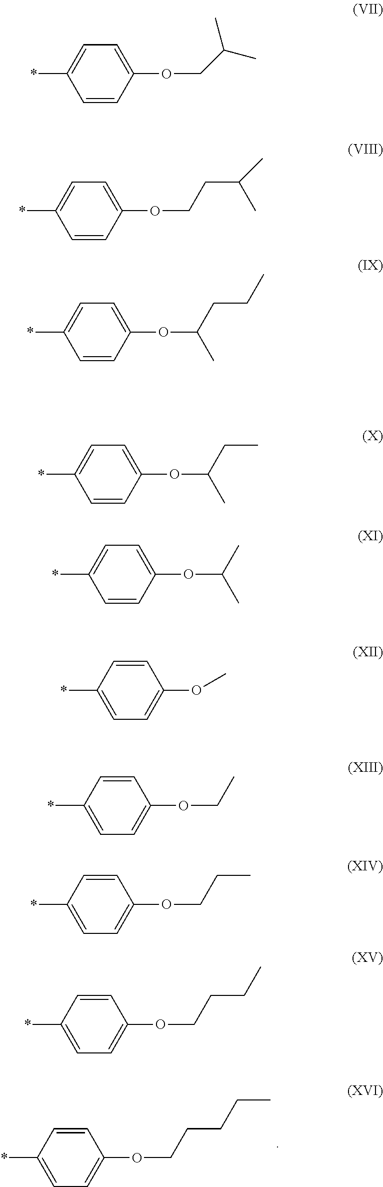

The fluorescent sensor compounds can fluoresce when exposed to light. In some examples, the fluorescent sensor compounds can fluoresce when exposed to ultraviolet light. in various examples, the ultraviolet light can have a wavelength of 100 nm to 400 nm. The ultraviolet light can be UVA (315-400, UVB (280-315), or UVC (100-280). When the fluorescent sensor compounds are exposed to THC or CBD, the fluorescence intensity can decrease. The sensitivity of the fluorescent sensor compound can depend on the magnitude of fluorescence intensity change when exposed to a given concentration of THC or CBD. In some examples, THC molecules can bind to the fluorescent sensor compound molecules. The binding affinity can be affected by π-π interactions between the aromatic ring of THC and the fluorescent sensor molecule, and also by dipole-dipole interactions between THC and the fluorescent sensor molecule. The dipole-dipole interactions can be strengthened by designing the fluorescent sensor compound with electron donor and electron acceptor groups as explained above. Larger numbers of electron donor groups and electron acceptor groups can provide greater sensitivity due to stronger interactions between THC and the fluorescent sensor compound. In some examples, the fluorescent sensor compound can have from 1 to 5 electron donor groups and from 1 to 5 electron acceptor groups. In further examples, the fluorescent sensor compound can have from 1 to 2 electron acceptor groups, from 1 to 4 electron donor groups, from 1 to 3 electron donor groups, or from 1 to 2 electron donor groups.

The higher fluorescence intensity can also allow for higher sensitivity in some examples. In various examples, all the electron acceptor groups in the fluorescent sensor compound can be benzothiadiazole.

The fluorescent sensor compounds can be described as oligomers, as opposed to polymers, because the compounds can be made up of a relatively small number of functional groups. In some examples, the fluorescent sensor compound can consist of 4 to 12 functional groups, where the functional groups include electron donor groups, electron acceptor groups, and end groups. The electron donor groups can be the fluorene groups as described above. The electron acceptor groups can be benzothiadiazole as described above. The end groups can be alkoxyphenyl groups as described above. In further examples, the total number of these functional groups in the fluorescent sensor compound can be from 4 to 10, 4 to 9, 5 to 9, 5 to 8, 5 to 7, 5 to 6, or 7 to 9. In further examples, the fluorescent sensor compound can have a molecular weight from about 700 g/mol to about 2500 g/mol.

The fluorescent compounds described herein can be prepared using synthetic methods described in the following description and Examples below.

### Synthesis Examples

Synthesis of the analogous compounds as in Formula (III), Formula (IV), Formula (V), and Formula (VI) can be achieved using the following guidelines. Other reactions may be used and this outline is not intended to be exclusive but rather exemplary.

1) synthesis of bromophenyl alkyl ether derivatives 1;

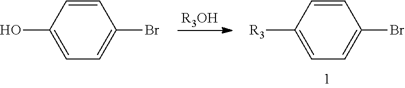

To a tetrahydrofuran solution (40 mL) of 4-bromophenol (20.0 mmol), alkyl alcohol (24.0 mmol), and triphenylphosphine (24.0 mmol), Diisopropyl azodicarboxylate (24.0 mmol) was dropwise added at 0° C. under Ar. Then the mixture was warmed up to room temperature and stirred overnight. After removal of the solvent under vacuum, the residue was purified by column chromatography on the silica gel (petroleum as the eluent) to afford bromophenyl alkyl ether derivatives 1.

2) Reaction of intermediate 1 with bis(pinacolato)diboron to obtain intermediate 2;

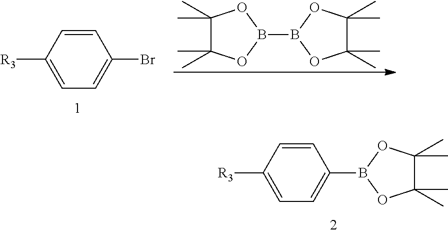

A mixture of intermediate 1 (1.0 eq), potassium acetate (3.0 eq), bis(pinacolato)diboron (1.2 eq), and Pd(dppf)Cl₂ (0.05 eq) in deoxygenated 1,4-dioxane (20 to 100 mL) was heated to 80° C. and stirred overnight under Ar. The solvent was evaporated under vacuum. The residue was poured into water (20 to 100 mL) and extracted with ethyl acetate (3*20 to 3*100 mL). The combined organic layer was washed with brine (saturated), dried over Na₂SO₄, and concentrated under vacuum. The residue was purified by column chromatography on the silica gel to afford intermediate 2.

3) Reaction of intermediate 2 with 2,7-dibromofluorene derivatives or 4,7-dibromobenzodizole derivatives to obtain intermediate 3;

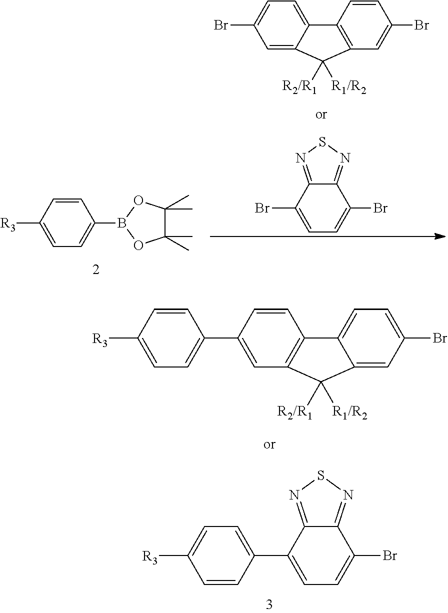

To a stirred solution of intermediate 2 (1.0 eq), 2,7-dibromofluorene derivatives (1.2 eq) or 4,7-dibromobenzodizole derivatives (1.2 eq) and tetrakis(triphenylphosphine)palladium (0.05 eq) in the mixed solvent with a ratio of 5:1 for 1,4-dioxane and water (20 to 100 mL), potassium carbonate (3.0 eq) was added. Then the mixture was heated to 80° C. and stirred overnight under Ar. The solvent was evaporated under vacuum. The residue was poured into water (20 to 100 mL) and extracted with ethyl acetate ((3*20 to 3*100 mL). The combined organic layer was washed with brine (saturated), dried over Na₂SO₄, and concentrated under vacuum. The residue was purified by column chromatography on the silica gel to afford intermediate 3.

4) Reaction of intermediate 3 with bis(pinacolato)diboron to obtain intermediate 4;

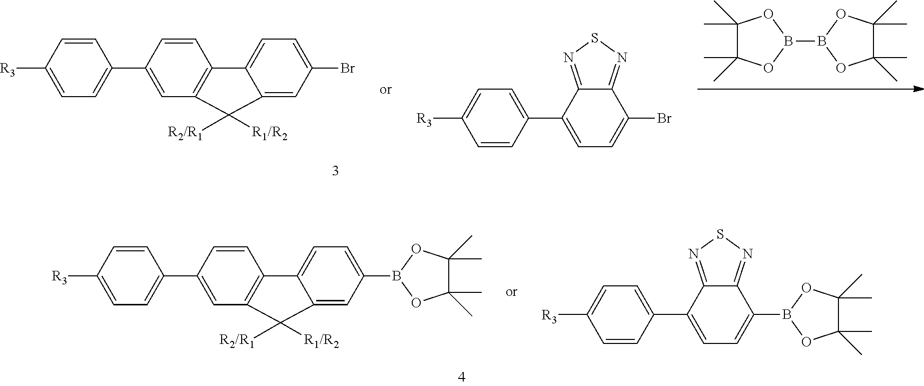

A mixture of intermediate 3 (1.0 eq), potassium acetate (3.0 eq), bis(pinacolato)diboron (1.2 eq), and Pd(dppf)Cl₂ (0.05 eq) in deoxygenated 1,4-dioxane (20 to 100 mL) was heated to 80° C. and stirred overnight under Ar. The solvent was evaporated under vacuum. The residue was poured into water (20 to 100 mL) and extracted with ethyl acetate (3*20 to 3*100 mL). The combined organic layer was washed with brine (saturated), dried over Na₂SO₄, and concentrated under vacuum. The residue was purified by column chromatography on the silica gel to afford intermediate 4.

5) The intermediate 2 is subjected to multiple above Suzuki Miyaura reactions to obtain the intermediate 5;

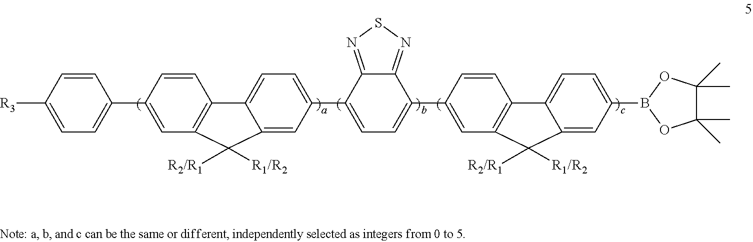

6) Reaction of intermediate 5 with 2,7-dibromofluorene derivatives or 4,7-dibromobenzodizole derivatives to obtain the analogous compound as Formula (III), Formula (IV), Formula (V), and Formula (VI).

To a stirred solution of intermediate 5 (2.4 eq), 2,7-dibromofluorene derivatives (1.0 eq) or 4,7-dibromobenzodizole derivatives (1.0 eq) and tetrakis(triphenylphosphine)palladium (0.10 eq) in the mixed solvent with a ratio of 5:1 for 1,4-dioxane and water (20 to 100 mL), potassium carbonate (5.0 eq) was added. Then the mixture was heated to 80° C. and stirred overnight under Ar. The solvent was evaporated under vacuum. The residue was poured into water (20 to 100 mL) and extracted with ethyl acetate ((3*20 to 3*100 mL). The combined organic layer was washed with brine (saturated), dried over Na₂SO₄, and concentrated under vacuum. The residue was purified by column chromatography on the silica gel to afford intermediate 3.

### Case 1

For the Formula (III), a=1, b=0, and c=0 in the molecular structure formula of the intermediate 5.

The synthesis route includes five steps: 1), 2), 3), 4) and 6). The dibrominated reactant in (3) was 2,7-dibromofluorene derivative and the dibrominated reactant in (6) was 4,7-dibromobenzodizole derivative. The feeding amount and operation were the same as those described in the general method.

### Case 2

For the Formula (IV), a=1, b=0, and c=1 in the molecular structure formula of the intermediate 5.

The synthesis route includes seven steps: 1), 2), 3), 4), 3), 4), and 6). The dibrominated reactant in the first (3) was 2,7-dibromofluorene derivative with the alkyl groups of R₂ and the dibrominated reactant in the second (3) was 2,7-dibromofluorene derivative with the alkyl groups of R₁. The dibrominated reactant in (6) was 4,7-dibromobenzodizole derivative. The feeding amount and operation were the same as those described in the general method.

### Case 3

For the Formula (V), a=0, b=1, and c=1 in the molecular structure formula of the intermediate 5.

The synthesis route includes seven steps: 1), 2), 3), 4), 3), 4), and 6). The dibrominated reactant in the first (3) was 4,7-dibromobenzodizole derivative and the dibrominated reactant in the second (3) was 2,7-dibromofluorene derivative with the alkyl groups of R₁. The dibrominated reactant in (6) was 2,7-dibromofluorene derivative with the alkyl groups of R₁. The feeding amount and operation were the same as those described in the general method.

### Case 4

For the Formula (VI), a=1, b=1, and c=1 in the molecular structure formula of the intermediate 5.

The synthesis route includes nine steps: 1), 2), 3), 4), 3), 4), 3), 4), and 6). The dibrominated reactants in the first, second, and third (3) were 2,7-dibromofluorene derivatives with the alkyl groups of R₂, 4,7-dibromobenzodizole derivatives, and 2,7-dibromofluorene derivatives with the alkyl groups of R₁ respectively. The dibrominated reactant in (6) was 2,7-dibromofluorene derivative with the alkyl groups of R₁. The feeding amount and operation were the same as those described in the general method.

### Sensor Compound Morphology,

In some examples, the fluorescent sensor compounds can form nanostructures such as nanofibrils, nanoparticles. In certain examples, the nanostructures can form partially through n-n interactions between the aromatic groups of adjacent molecules of the fluorescent sensor compound. A sufficient amount of the fluorescent sensor compound can form porous structures made up of these nanostructures. In some examples, nanofibrils can form a porous net structure. This structure can be useful for detecting THC and CBD in the gas phase, because THC and CBD molecules can penetrate into the net structure. Additionally, the net structure can have a high surface area, which increases the likelihood that a THC or CBD molecule will interact with the fluorescent sensor compound. However, if the porous net structure is formed as a thick layer, then at some depth within the layer it can become difficult to detect changes in fluorescence caused by THC or CBD molecules. For example, fluorescence changes of nanofibrils of the fluorescent sensor molecules deep in the film may be obscured by other nanofibrils closer to the surface. Therefore, above a certain thickness it may not be useful to increase the thickness of a porous film of the fluorescent sensor compound. In some examples, a porous film of the fluorescent sensor compound can have a thickness from about 1 micrometer to about 100 micrometers, or from about 10 micrometers to about 100 micrometers, or from about 50 micrometers to about 100 micrometers, or from about 1 micrometer to about 10 micrometers, or from about 1 micrometer to about 50 micrometers.

In other examples, the fluorescent sensor compound can be in the form of a thin, non-porous film. The thin, non-porous film can have a thickness from about 100 nanometers to about 10 micrometers, or from about 100 nanometers to about 5 micrometers, or from about 100 nanometers to about 1 micrometer, or from about 100 nanometers to about 500 nanometers, or from about 500 nanometers to about 1 micrometer, or from about 500 nanometers to about 5 micrometers, or from about 500 nanometers to about 10 micrometers, or from about 1 micrometer to about 5 micrometers, or from about 1 micrometer to about 10 micrometers, or from about 5 micrometers to about 10 micrometers. Whether the fluorescent sensor compound is deposited as a porous film or as a non-porous film, the compound can be deposited on a substrate. Substrates can include glass, silicon, quartz, and others. The substrate can have a variety of shapes, such as a flat plate, a pair of flat surfaces that form a channel, a tube, baffles, a screen or grate, or other shapes. In some examples, the substrate and the film of the fluorescent sensor compound can be designed to provide contact between the fluorescent sensor compound and a flow of exhaled breath or other gas.

### Fluorescent Sensors for Detecting Marijuana

The present disclosure also describes fluorescent sensor for detecting marijuana. The fluorescent sensors can include a fluorescent sensor compound as described above. The sensor can also include a fluorescence detector oriented to detect a fluorescence response from the fluorescent sensor compound. In some examples, the fluorescence response can be a reduction in fluorescence intensity upon exposure to THC or CBD.

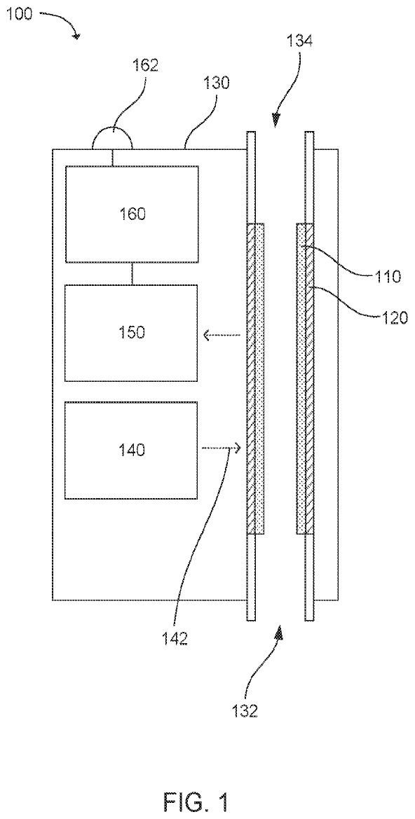

FIG. 1 is a schematic diagram of an example fluorescent sensor 100. This sensor includes a porous film 110 of a fluorescent sensor compound on a substrate 120. In this example, the substrate is a tube (cross-section of the tube is shown) and the film of the fluorescent sensor compound is deposited on the inside surface of the tube. A cross-section of the tube is shown in the figure. The sensor also includes a housing 130. The housing includes a breath inlet 132 and an outlet 134. In this example, the housing can be shaped as a small rectangular box with a breath inlet hole for a user to breath into and a breath outlet hole for the breath to exit after passing through the sensor. The breath inlet and outlet are connected to the tube containing the fluorescent sensor compound so that exhaled breath can pass the film of fluorescent sensor compound. The sensor also includes an excitation light source 140 and a fluorescence detector 150. In this example, the tube substrate is transparent so that light 142 can be pass though the tube. The light from the excitation light source can pass through the tube to excite the fluorescent sensor compound. Light produced by fluorescence can also pass through the tube to be detected by the detector. A signal from the detector is sent to a comparator 160. The comparator is also connected to an indicator 162. Connections between the comparator and the fluorescence detector and the indicator are shown as solid lines. The comparator can be configured to compare the fluorescence intensity detected by the detector with a threshold value, and if the fluorescence intensity falls below the threshold upon exposure of the sensor to a sample then this can suggest that the sample contains THC or CBD. The comparator can produce a signal using the indicator if THC or CBD is detected. The indicator can include a visible indicator, such as an LED light, or an audible indicator such as an alarm, buzzer, or beeper. In alternative examples, the detector can be connected to an external module such as a personal computer, which can display test results in a variety of ways.

The example shown in FIG. 1 is designed to directly detect THC or CBD in exhaled breath. In other examples, the sensor can include a condensing surface. The sensor housing can direct exhaled breath to the condensing surface to form a breath condensate on the condensing surface. The breath condensate can then be heated to evaporate the breath condensate. The vapor can by directed to contact the fluorescent sensor compound to detect THC or CBD in the vapor. These compounds can be more concentrated in the breath condensate compared to freshly exhaled breath. Therefore, in some cases the compounds can be detected with a greater sensitivity when the breath is condensed in this way. In some examples, the sensor can include a heater to heat the breath condensate. In other examples, a two part sensor can be made, in which a user breathes into one part containing a condensing surface to collect a breath condensate on the condensing surface, and then the condensing surface can be loaded or connected to a second part of the sensor, where the second part includes the fluorescent sensing compound for detecting THC or CBD. In certain examples, the condensing surface can be incorporated in a disposable cartridge for one-time-use. The condensing surface can be a pre-concentration membrane similar to membranes used for solid-phase extraction. The membrane can be configured to interact with THC and/or CBD so that these compounds are captured from the exhaled breath.

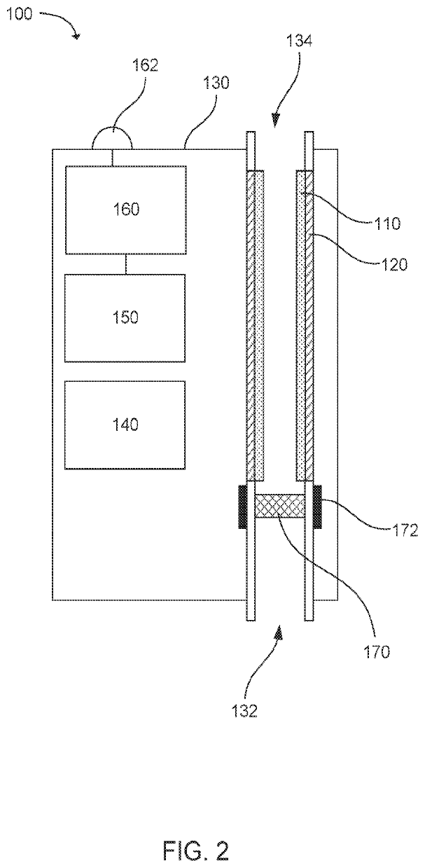

FIG. 2 is a schematic diagram of another example fluorescent sensor 100. This example includes a housing 130 with a breath inlet 132 and breath outlet 134. The breath inlet leads to a condensing surface 170, which in this example is a pre-concentration membrane. The breath passes through the pre-concentration membrane and THC and CBD in the breath are captured by the membrane. After a user has breathed into the sensor, a heater 172 can be used to heat the membrane and vaporize the condensed components of the breath. In this example, the heater is an electric heating element shaped as a ring surrounding the pre-concentration membrane (a cross-section of the ring-shaped heating element is shown). The vaporized material can then contact the film of fluorescent sensor compound 110 on the substrate 120 as in the previous example. In some examples, the vaporized material can diffuse to the film of fluorescent sensor compound, and in other examples the sensor can include a pump to pump the vaporized material to the film of fluorescent sensor compound. This example also includes an excitation light source 140, a fluorescence detector 150, a comparator 160, and an indicator 162 as in the previous example.

In some examples, the sensors described herein can detect THC or CBD from exhaled breath containing the THC or CBD at concentrations with a limit of detection from about 0.0008 parts per billion by volume (ppbv) to about 0.2 ppbv. In certain examples, the sensor can utilize a condensing surface to condense and pre-concentrate compounds from the exhaled breath. This can increase the sensitivity of detection. In some such examples, the limit of detection can be from about 0.0008 ppbv to about 0.08 ppbv. In other examples, the sensor can detect THC or CBD directly from exhaled breath. In some such examples, the limit of detection can be from about 0.02 ppbv to about 0.2 ppbv. Typical concentrations of THC in the breath of a user within three hours after using marijuana can range from about 0.004 ppbv to about 0.07 ppbv in some examples. The total amount of THC or CBD present in the sensor during detection can be on the order of nanograms to picograms. In some examples, the sensor can have a limit of detection based on a total amount of THC or CBD inside the sensor, where the limit of detection is from 1 picogram to 100 nanograms, or from 10 picograms to 10 nanograms, 100 picograms to 10 nanograms, 500 picograms to 10 nanograms, 1 nanogram to 10 nanograms, 1 picogram to 1 nanogram, 1 picogram to 500 picograms, 1 picogram to 100 picograms, or 10 picograms to 100 picograms.

The sensors described herein can be reusable or can be manufactured to be disposable. When configured to be disposable, the sensor can be made of inexpensive materials including some or all of the materials being biodegradable.

### Methods of Detecting Marijuana Breath Markers

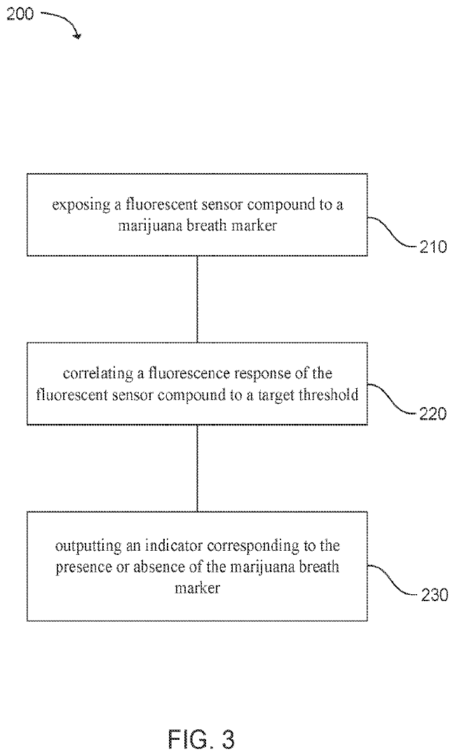

The present disclosure also describes methods of detecting marijuana breath markers. in some examples, the marijuana breath marker can be THC or CBD. FIG. 3 is a flowchart illustrating one example method 200 of detecting a marijuana breath marker. The method includes: exposing a fluorescent sensor compound to a marijuana breath marker 210; correlating a fluorescence response of the fluorescent sensor compound to a target threshold 220; and outputting an indicator corresponding to the presence or absence of the marijuana breath marker 230. In some examples, the breath sample can be directly exhaled breath, a breath condensate, a pre-concentrated breath, or a combination thereof.

In some examples, the fluorescence response to THC or CBD can include a drop in fluorescence intensity upon exposure to the THC or CBD. If the fluorescence intensity drops below a target threshold then this can indicate the presence of THC or CBD. The target threshold can be determined as a percentage drop based on a baseline fluorescence intensity. In some cases, a sensor can be pre-tested with known samples of THC or CBD to determine a threshold fluorescence response that has a sufficient likelihood of indicating the presence of THC or CBD, as opposed to background noise. In certain examples, the threshold fluorescence response can be selected to represent a 95% likelihood that THC or CBD is present, or a 99% likelihood, or a 99.9% likelihood. The sensor can be capable of detecting very small drops in fluorescence intensity, and therefore can be capable of accurately detecting small concentrations of THC and CBD. In some examples, the threshold drop in fluorescence intensity to indicate the presence of THC or CBD can be from about 0.02% to about 1%, or from about 0.02% to about 0.5%, or from about 0.02% to about 0.2%, or from about 0.02% to about 0.1%, or from about 0.02% to about 0.05%, or from about 0.05% to about 1%, or from about 0.05% to about 0.5%, or from about 0.05% to about 0.2%, or from about 0.05% to about 0.1%, or from about 0.1% to about 1%, or from about 0.1% to about 0.5%, or from about 0.1% to about 0.2%.

### EXAMPLES

### Example 1

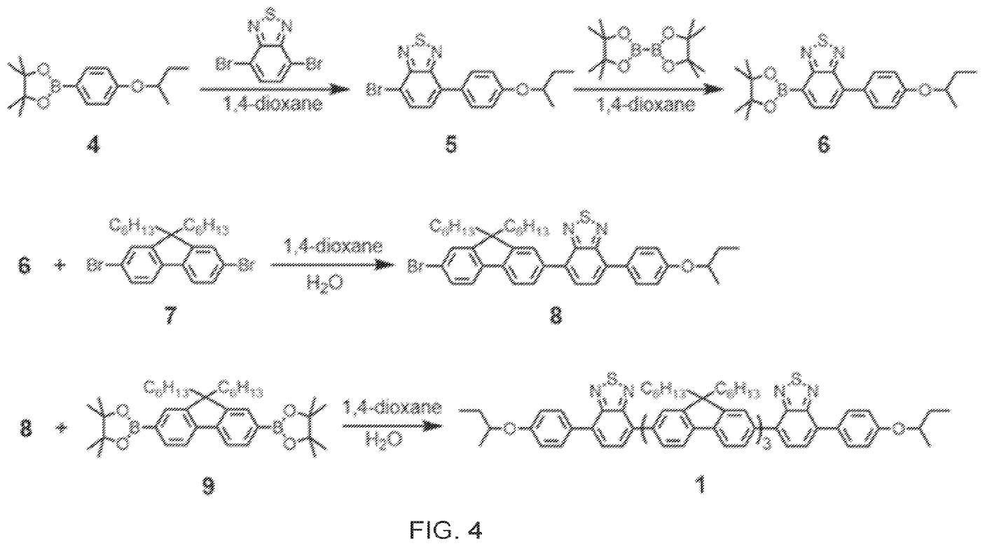

FIG. 4 shows a scheme of an example synthesis route for an example fluorescent sensor compound. This scheme is referred to as scheme S1 In this scheme, molecule 1 is the example fluorescent sensor compound. This molecule is named TFT-C6, or using IUPAC naming, the compound is 7,7′-(9,9,9′,9′,9″,9″-hexahexyl-9H,9′H,9″H-[2,2′:7′,2″-terfluorene]-7,7″-diyl)bis(4-(4-sec-butoxy)phenyl)benzo[c][1,2,5]thiadiazole). Note that the side group attached to the fluorene moiety can be adjusted in different length of alkyl chains, ranging from C2 to C8. As currently tested, both C6 (molecule 1 shown in the figure here) and C4 (molecule 2 shown below in Scheme S2) substituted molecules as fabricated as thin film demonstrate effective fluorescent sensor response towards THC and CBD.

Molecules 4 and 9 were synthesized by following the previously reported method (C. Qiu, X. Liu, C. Cheng, Y. Gong, W. Xiong, Y. Guo, C. Wang, J. Zhao, Y. Che, Anal. Chem. 2019, 91, 6408, which is incorporated herein by reference). 4-bromo-7-(4-(sec-butoxy)phenyl)benzo[c][1,2,5]thiadiazole (5). To a stirred solution of molecule 4 (1.5 g, 5.43 mmol), 4,7-dibromobenzo[c][1,2,5]thiadiazole (1.9 g, 6.5 mmol), and tetrakis(triphenylphosphine)palladium (311 mg, 0.27 mmol) in deoxygenated 1,4-dioxane (40 mL), aqueous solution of potassium carbonate (7 mL, 2.0 M) was added. Then the mixture was heated to 80° C. and stirred overnight under Ar. The solvent was evaporated under vacuum. The residue was poured into water (50 mL) and extracted with ethyl acetate (3*40 mL). The combined organic layer was washed with brine (saturated), dried over Na₂SO₄, and concentrated under vacuum. The residue was purified by column chromatography on the silica gel (petroleum:dichloromethane=10:1 as the eluent) to afford molecule 5 (0.87 g, 2.40 mmol, 44% yield).

Compound 5 was confirmed by ¹H NMR (400 MHz, CDCl₃), finding the following peaks: δ 7.84 (d, J=2.8 Hz, 1H), 7.82 (d, J=7.6 Hz, 2H), 7.51 (d, J=7.6 Hz, 1H), 7.03 (dt, J=8.8 Hz, 2.8 Hz, 2H), 4.43-4.33 (m, 1H), 1.82-1.65 (m, 2H), 1.35 (d, J=6.0 Hz, 3H), 1.01 (t, J=7.2 Hz, 3H).

Molecule 6 is 4-(4-(sec-butoxy)phenyl)-7-(4,4,5,5-tetramethyl-1,3,2-dioxaborolan-2-yl)benzo[c][1,2,5]thiadiazole. To form this molecule, a mixture of molecule 5 (0.87 g, 2.40 mmol), potassium acetate (708 mg, 7.20 mmol), bis(pinacolato)diboron (731 mg, 2.88 mmol), and Pd(dppf)Cl₂ (88 mg, 0.12 mmol) in deoxygenated 1,4-dioxane (20 mL) was heated to 80° C. and stirred overnight under Ar. The solvent was evaporated under vacuum. The residue was poured into water (30 mL) and extracted with ethyl acetate (3*20 mL). The combined organic layer was washed with brine (saturated), dried over Na₂SO₄, and concentrated under vacuum. The residue was purified by column chromatography on the silica gel (petroleum dichloromethane=5:1 as the eluent) to afford molecule 6 (610 mg, 1.49 mmol, 62% yield).

Compound 6 was confirmed by ¹H NMR (400 MHz, CDCl₃), finding the following peaks: δ 8.23 (d, J=4.0 Hz, 1H), 7.91 (d, J=8.0 Hz, 2H), 7.65 (d, J=4.0 Hz, 1H), 7.03 (7.04, J=80 Hz, 21H), 4.39 (q, J=80 Hz, 1H), 1.83-1.61 (m, 2H), 1.45 (s, 3H), 1.34 (t, J=8.0 Hz, 12H), 1.01 (t, J=8.0 Hz, 3H).

Molecule 8 is 4-(7-bromo-9,9-dihexyl-9H-fluoren-2-yl)-7-(4-(sec-butoxy)phenyl)benzo[c][1,2,5]thiadiazole. To a stirred solution of compound 6 (368 mg, 0.90 mmol), 2,7-dibromo-9,9-hexyl-9H-fluorene (7) (530 mg, 1.07 mmol), and tetrakis(triphenylphosphine)palladium (51.8 mg, 0.05 mmol) in deoxygenated 1,4-dioxane (20 mL), aqueous solution of potassium carbonate (4 mL, 2.0 M) was added. Then the mixture was heated to 80° C. and stirred overnight under Ar. The solvent was evaporated under vacuum. The residue was poured into water (30 mL) and extracted with ethyl acetate (3*30 mL). The combined organic layer was washed with brine (saturated), dried over Na₂SO₄, and concentrated under vacuum. The residue was purified by column chromatography on the silica gel (petroleum:dichloromethane=2:1 as the eluent) to afford molecule 8 (310 mg, 0.44 mmol, 49% yield).

Compound 8 was confirmed by ¹H NMR (400 MHz, CDCl₃), finding the following peaks: δ 8.01-7.98 (m, 1H), 7.94-7.92 (m, 3H), 7.84-7.75 (m, 2H), 7.81 (d, J=8.0 Hz, 1H), 7.61 (d, J=8.0 Hz, 1H), 7.50-7.47 (m, 2H), 7.07 (d, J=8.8 Hz, 2H), 4.48-4.35 (m, 1H), 2.06-1.96 (m, 4H), 1.89-1.62 (m, 2H), 1.38 (d, J=4.0 Hz, 3H), 1.19-1.05 (m, 12H), 1.02 (t, J=8.0 Hz, 3H), 0.79-0.74 (m, 10H).

Compound 1 is 7,7′-(9,9,9′,9′,9″,9″-hexahexyl-9H,9′H,9″H-[2,2′,7′,2″-terfluorene]-7,7″-diyl)bis(4-(4-sec-butoxy)phenyl)benzo[c][1,2,5]thiadiazole). To a stirred solution of compound 8 (230 mg, 0.33 mmol), compound 9 (87.8 mg, 0.15 mmol), and tetrakis(triphenylphosphine)palladium (19 mg, 0.017 mmol) in deoxygenated 1,4-dioxane (20 mL), aqueous solution of potassium carbonate (3 mL, 2.0 M) was added. Then the mixture was heated to 80° C. and stirred overnight under Ar. The solvent was evaporated under vacuum. The residue was poured into water (20 mL) and extracted with ethyl acetate (3*20 mL). The combined organic layer was washed with brine (saturated), dried over Na₂SO₄, and concentrated under vacuum. The residue was purified by column chromatography on silica gel (petroleum:dichloromethane=1:1 as the eluent) to afford compound 1 (119 mg, 0.08 mmol, 51% yield). The resulting target compound was confirmed by ¹H NMR and MALDI-MS as below.

TFT-C6 (i.e., compound 1) was confirmed by ¹H NMR (400 MHz, CDCl₃), finding the following peaks: 8.05 (d, J=8.0 Hz, 2H), δ 7.96-7.77 (m, 14H), 7.78 (d, J=8.8 Hz, 2H), 7.71-7.67 (m, 8H), 7.08 (d, J=8.8 Hz, 4H), 4.22 (q, J=6.0 Hz, 2H), 2.21-2.08 (m, 12H), 1.91-1.62 (m, 4H), 1.39 (d, J=4.0 Hz, 6H), 1.68-1.23 (m, 36H), 1.05 (t, J=8.0 Hz, 6H), 0.93-0.82 (m, 12H), 0.80-0.71 (n, 18H). MS (MALDI-MS). [M]⁺ calculated for C₁₀₇H₁₂₆N₄O₂S₂ 1563.9, found 1563.7.

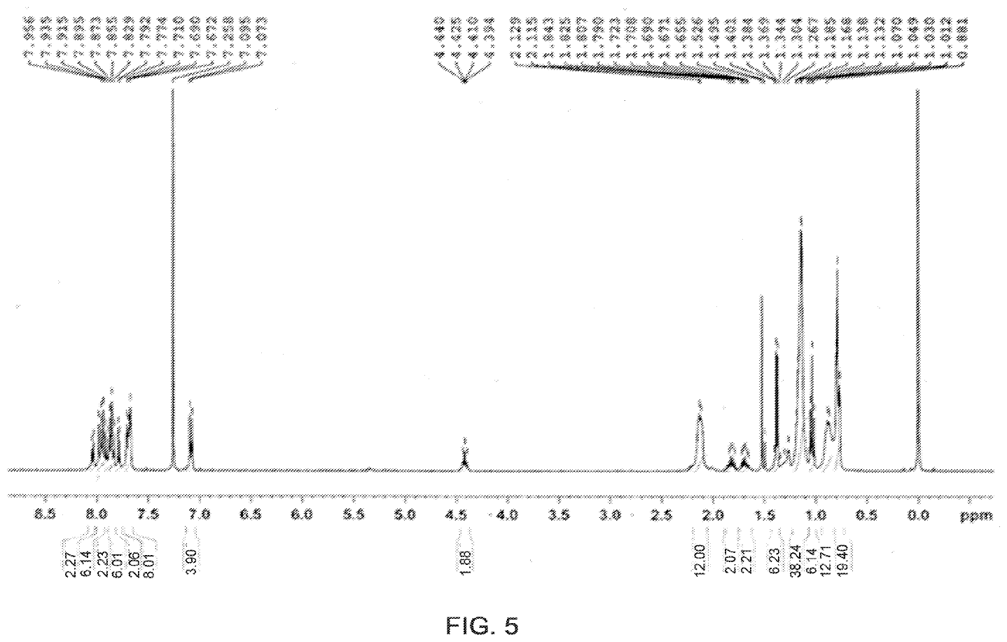

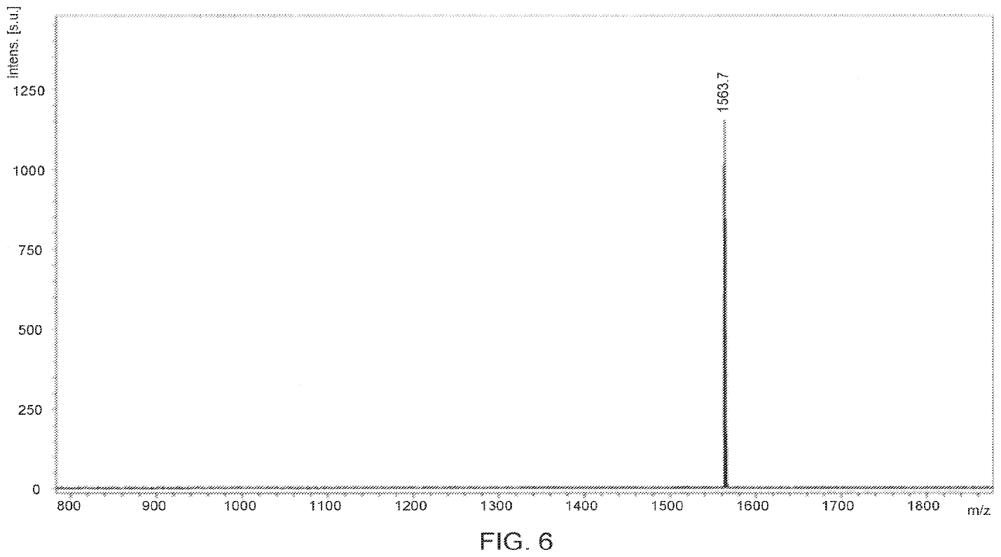

The NMR and MS spectra of TFT-C6 are shown in FIGS. 5 and 6.

### Example 2

Molecule 2 is named TFT-C4. This molecule was synthesized by following the previously reported method, but the alkyl chain length is changed. The structure of molecule 2 is shown in Formula (XVII) below:

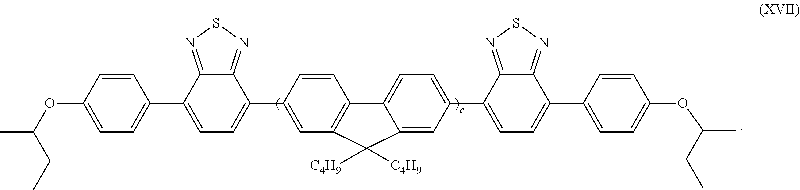

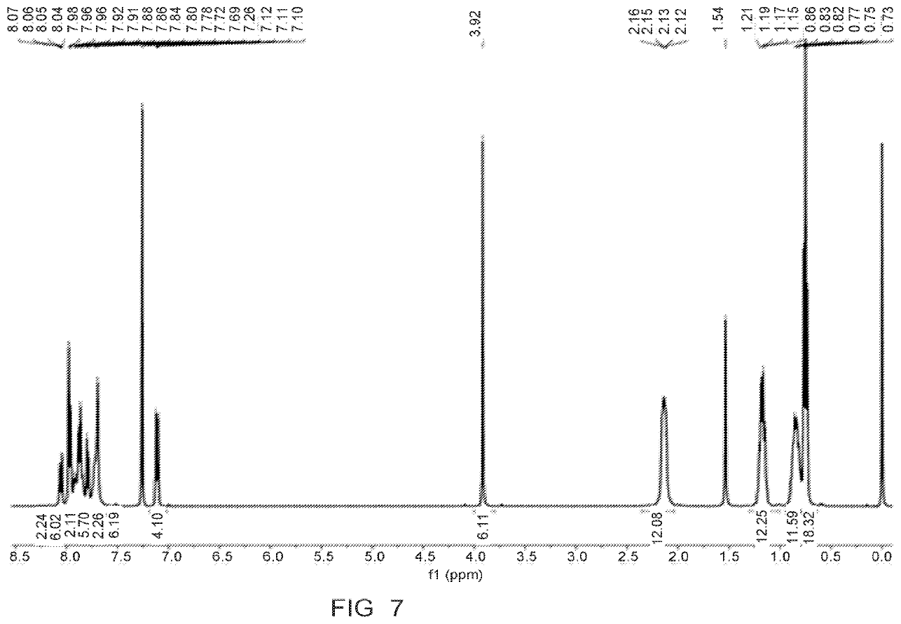

As shown in FIG. 7, TFT-C4 was confirmed using ¹H NMR (400 MHz, CDCl₃), finding the following peaks: 8.07-8.04 (d, J=8.0 Hz, 2H), δ 7.98-7.80 (m, 14H), 7.78 (d, J 8.8 Hz, 2H), 7.72-7.67 (m, 8H), 7.12-7.10 (d, J=8.8 Hz, 4H), 3.92 (s, 6H), 2.16-2.12 (m, 12H), 1.21-1.15 (m, 12H), 0.86-0.82 (m, 12H), 0.77-0.73 (m, 18H).

### Example 3

Results of gas phase detection of THC and CBD, and potential interferents present in breath and local environment were tested. First, the TFT-C6 molecules were caused to self-assemble by the following procedure. Inject 0.1 mL CHCl₃ solution with TFT-C6 (6 mg/mL) into a vial, and then add 2 mL methanol to the same vial aging for 4 h. The resulting assemblies will be suspended in the solutions for casting inside the quartz tubes.

Fluorescent sensing was then performed using the following procedure. 10 μL of the aggregates suspension in methanol was cast inside a quartz tube about 0.3 cm away from the air-inlet. The remaining solvent was removed from the quartz tube by a capillary. The tube was then dried by a blower for 30 s at first and then in vacuum for 2 h to remove all the solvents inside. 0.2 mL THC or CBD methanol solution (1 mg/mL) was transferred into a 40 mL vial, followed by drying under vacuum for 4 h to remove the solvents. The vial was placed in a clean oven at different temperatures for 30 min to get the saturated vapor inside the vial. The head space sample of the vial was then pumped into the sensor tube (at flow rate of 150 sccm) for 10 s for sensor testing.

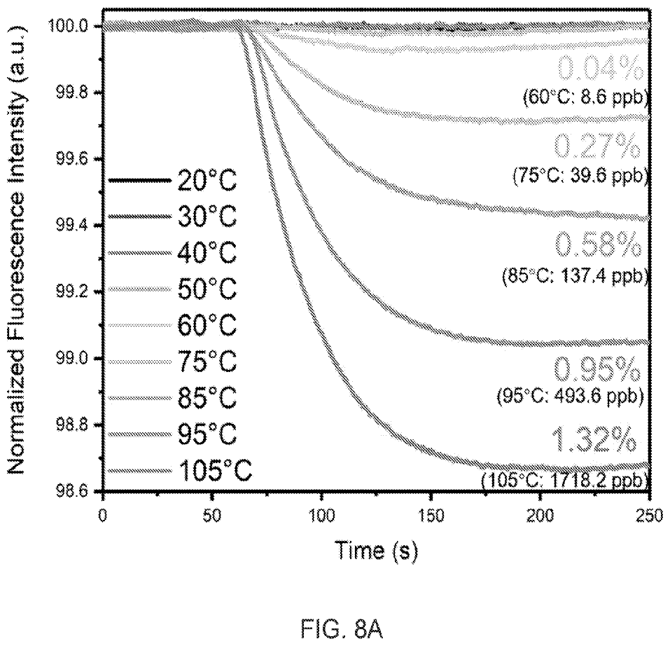

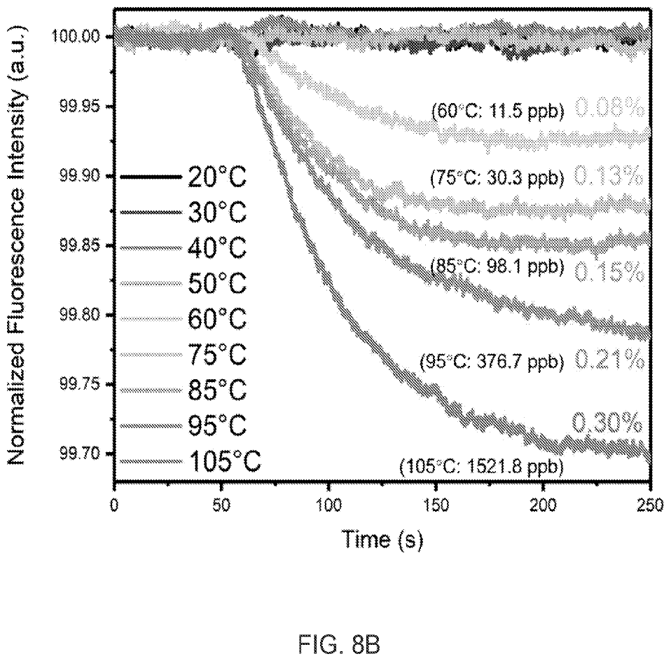

FIG. 8A shows fluorescence quenching response to THC vapor at different temperatures. FIG. 8B shows fluorescence response to CBD vapor at different temperatures. (Predicted vapor pressure of THC under different temperatures: 60° C.: 8.6 ppb, 75° C.: 39.6 ppb, 85° C.: 137.4 ppb, 95° C.: 493.6 ppb, 105° C.: 1718.2 ppb; predicted vapor pressure of CBD under different temperatures: 60° C.: 11.5 ppb, 75° C.: 30.3 ppb, 85° C.: 98.1 ppb, 95° C.: 376.7 ppb, 105° C.: 1521.8 ppb).

The limit of detection (LOD) is determined by the following equations (500 points were chosen to calculate the root-mean-square value of the noise: RMS_{Noise}):

$$\begin{matrix}LOD = \frac{{ak ( 3 \times {RMS}_{Noise} )}^{n}}{1 + {k ( 3 \times {RMS}_{Noise} )}^{n}} \\ {RMS}_{Noise} = \sqrt{\frac{\sum {( {I}_{i} - {I}_{c} )}^{2}}{N \times {( {I}_{0} )}^{2}}}\end{matrix}$$

Here, Iᵢ and I_{c} represent experimental and corrected fluorescence intensity values, respectively. I₀ is the fluorescence intensity value when time is 0. N represents the number of data points (500 points were chosen to calculate).

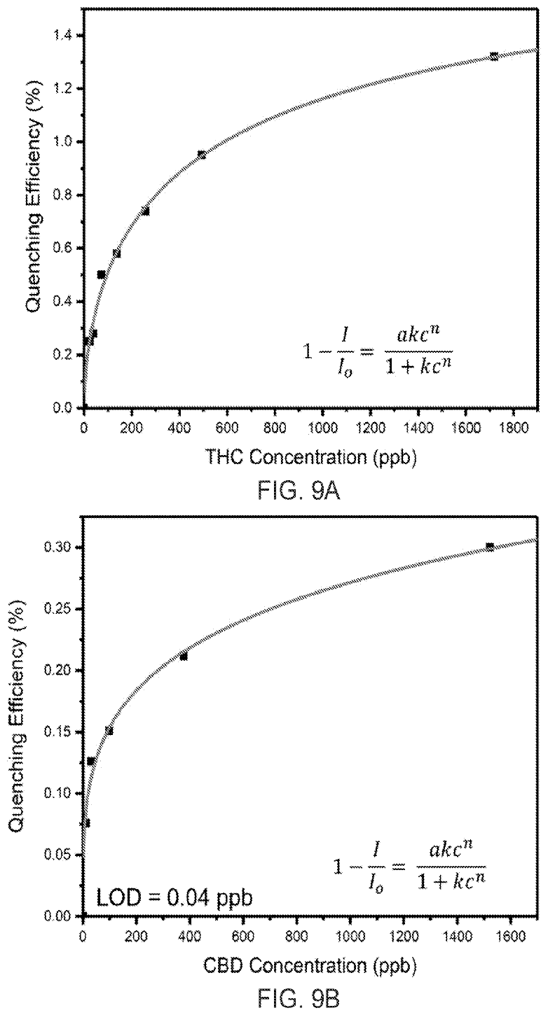

FIGS. 9A-9B show fitting curves and equation of the fluorescence quenching response obtained for (a) THC and (b) CBD at different vapor pressures (temperatures). The exponent “n” is a fitting parameter indicating the adjustment of Langmuir adsorption model due to the deviation from the ideal complete monolayer adsorption on surface. The calculated LOD of THC and CBD are 0.22 ppb and 0.04 ppb, respectively.

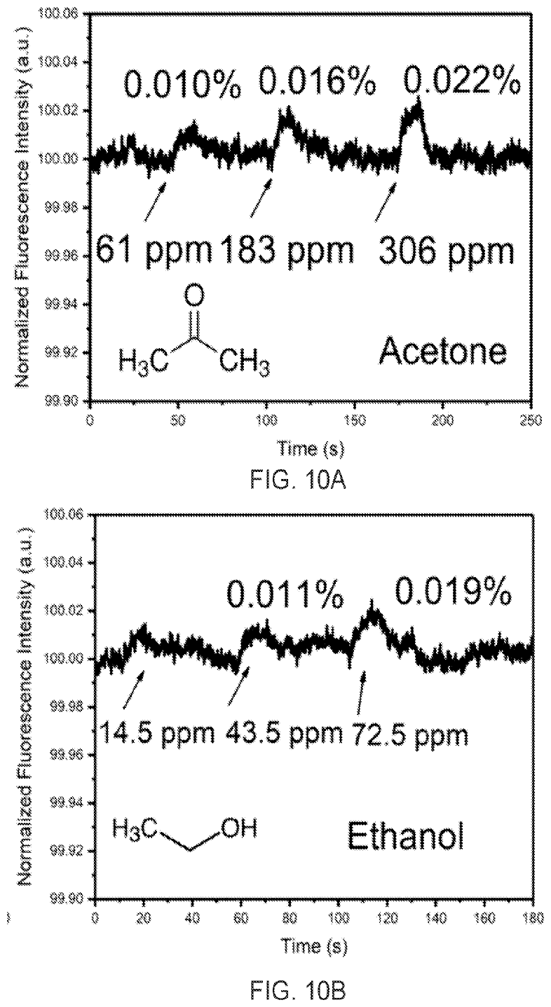

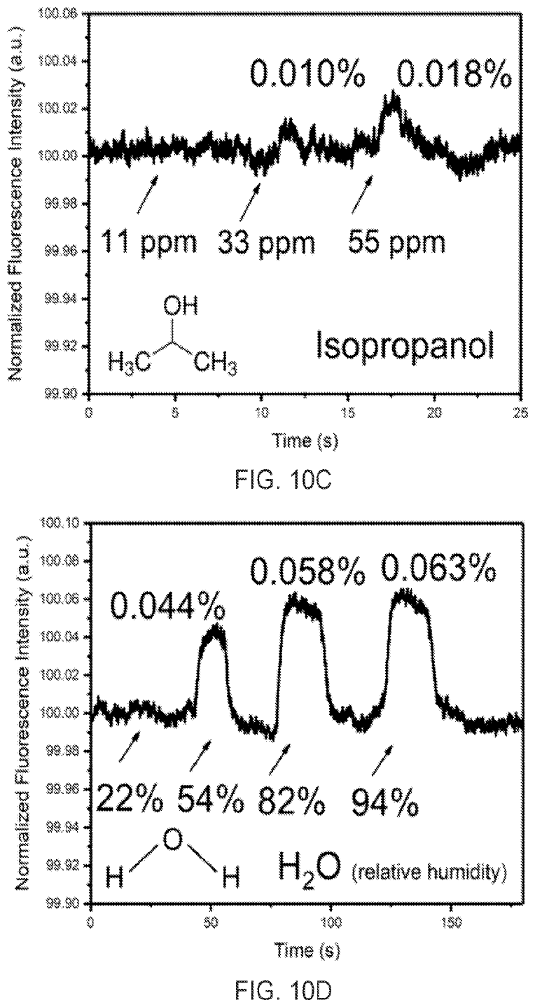

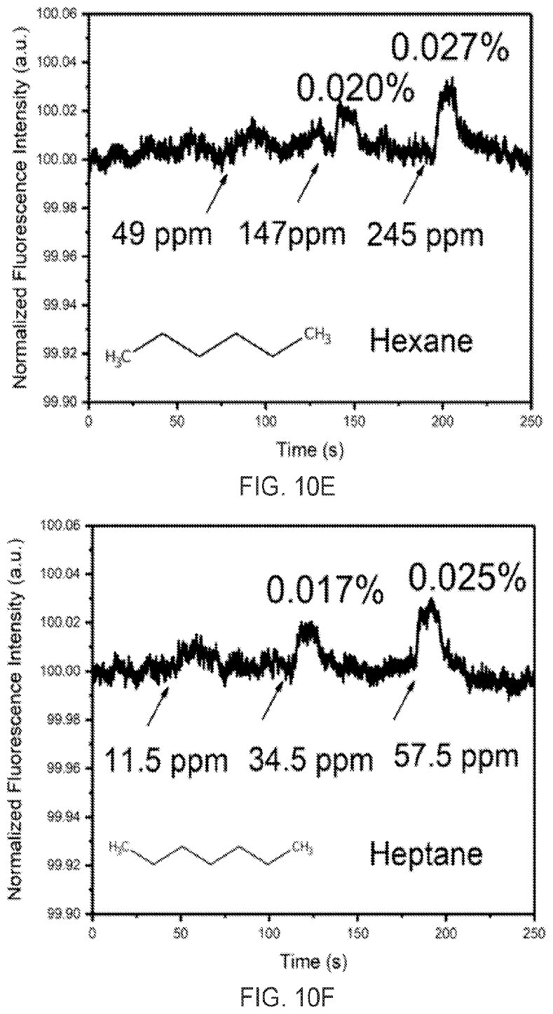

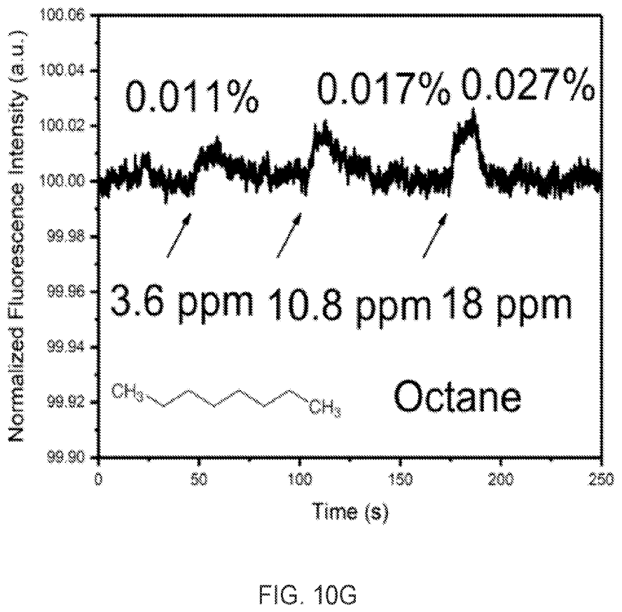

FIGS. 10A-G shows the fluorescence responses to the common potential interferents: (a) acetone (b) ethanol (c) isopropanol (d) water (relative humidity) (e) hexane (f) heptane (g) octane. All tested under the same conditions as performed for THC and CBD.

### Example 4

Trace level detection of THC and CBD on substrate was also tested. The compounds TFT-C6 and TFT-C4 were self-assembled to form aggregates by injecting 0.1 mL of the chloroform solution of TFT-C6 and TFT-C4 (both 6 mg/mL) into a vial containing 1 mL, of methanol and aging for 1 day at room temperature. The resulting assemblies that were suspended in solution could be cast onto various substrate and into a quartz tube.

Optical chambers for fluorescent sensing were prepared by casting 10 μL of the aggregate suspension in methanol inside a quartz tube about 0.3 cm away from the air-inlet. The remaining methanol was removed from the quartz tube by a capillary. Then, the resulting aggregates inside the quartz tube were dried for 1 min by a blower. Fluorescence quenching experiments by analytes were performed on a prototype detection device, which used a 380 nm LED lamp as the excitation light source. After a diluted solution of analytes (e.g. THC) in methanol was deposited onto a polytetrafluoroethylene (PTFE) film and dried in air, the PTFE film was inserted into the detector where the sample was vaporized at 170° C. by a thermal desorber and pumped into the optical chamber containing the sensing materials (air pump rate, 200 mL min⁻¹). Notably, the temperature of the optical chamber was ca. 60° C. during the detection process.

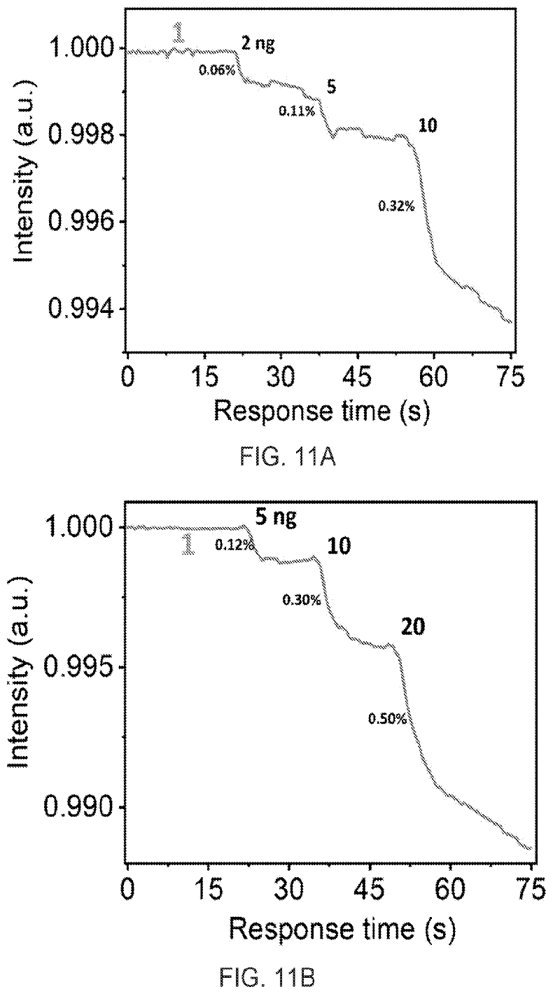

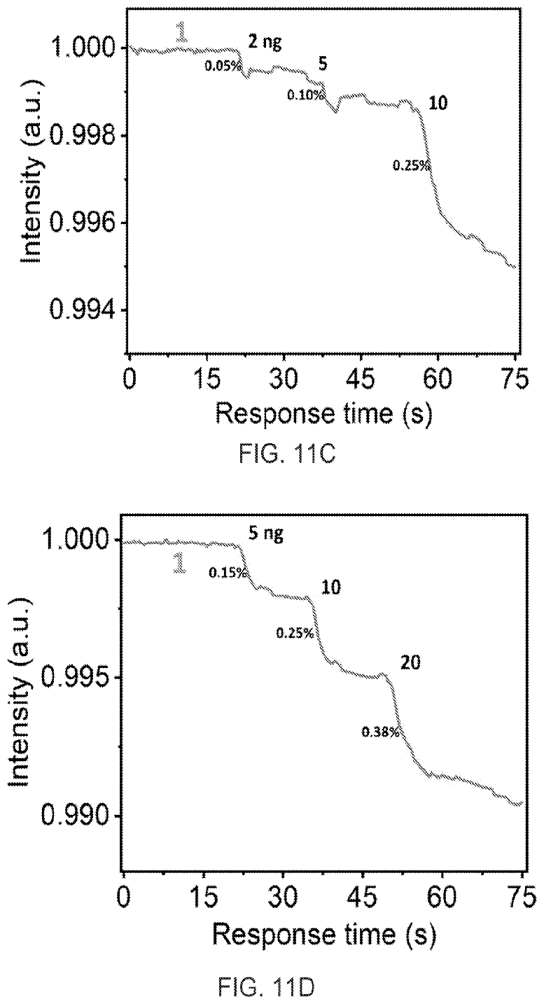

FIGS. 11A-11B show the fluorescence responses of (a) TFT-C6 and (b) TFT-C4 thin films towards 1, 2, 5, and 10 ng of THC deposited on a substrate under 170° C. instant heating, which was believed to be sufficient to vaporize all the solid sample. The second graph in each figure shows the results of repeating the same experiment with 5 ng, 10 ng, and 20 ng of THC.

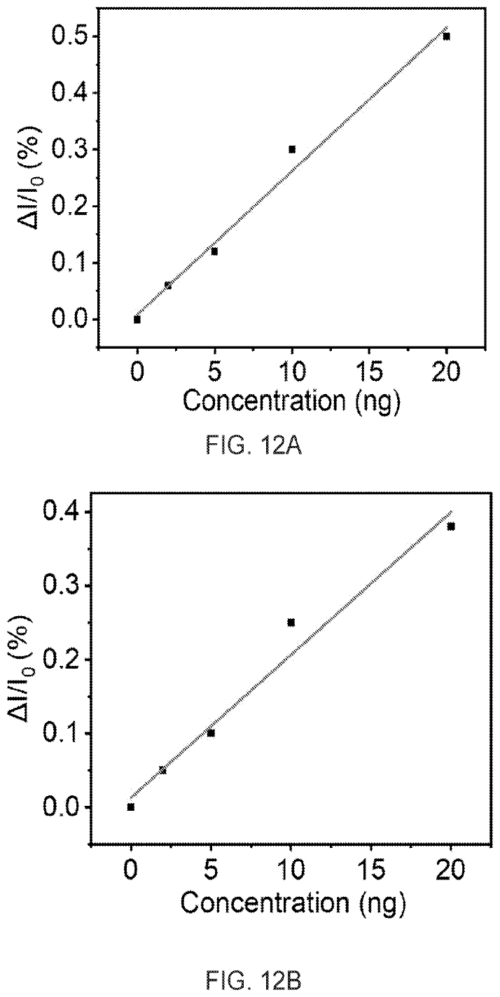

FIGS. 12A-12B show the linear fitting curves for LOD calculation of fluorescence responses of (a) TFT-C6 and (b) TFT-C4 towards vaporized THC.

The limit of detection (LOD) is determined by the following equations:

$$\begin{matrix}LOD = 3 \times {RMS}_{Noise} / slope \\ {RMS}_{Noise} = \sqrt{\frac{\sum {( {I}_{i} - {I}_{c} )}^{2}}{N \times {( {I}_{0} )}^{2}}}\end{matrix}$$

The LOD of TFT-C6 and TFT-C4 was estimated as 0.36 ng and 0.38 ng, respectively.

Another fluorescent sensor compound was also synthesized, having the structure shown

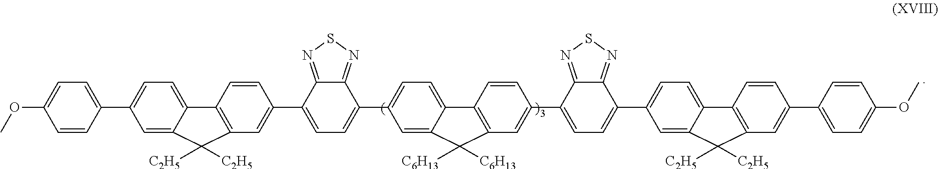

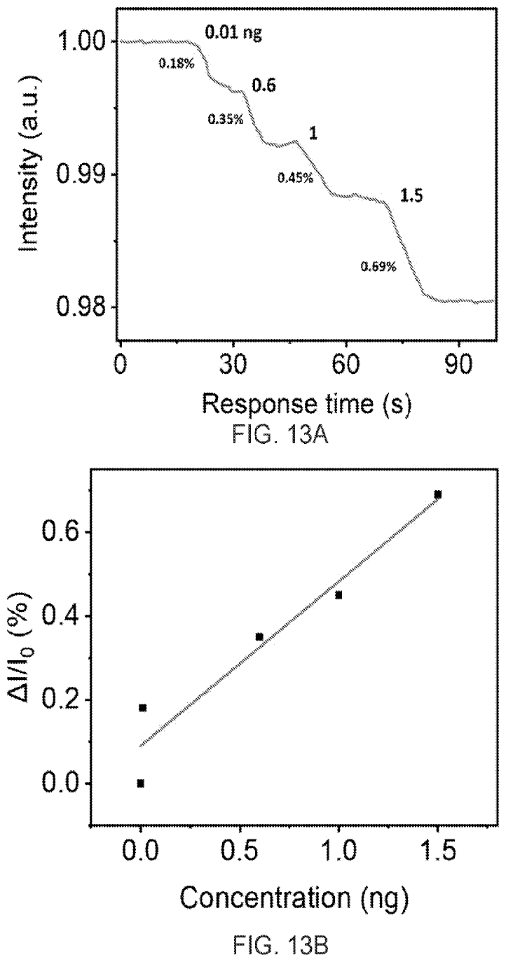

This compound was deposited in a quartz tube as in the previous experiments, and this compound was also tested by depositing THC in amounts of 0.01 ng, 0.6 ng, 1 ng, and 1.5 ng on a substrate and heating the THC at 170° C. The fluorescence response is shown in FIG. 13A. FIG. 13B shows the linear fitting curve for LOD calculation of the fluorescence responses using the same equations as used above. The LOD of this fluorescent sensor compound was estimated as 0.0013 ng, or 1.3 pg.

### Example 5

Testing of THC condensed with saliva shows the feasibility of detecting THC in real breath sample wherein moisture and many other compounds and bio-species present may interfere the sensor response. The sensor response was tested for 1 micro-liter saliva deposited on a PTFE strip. Further, the sensor response was tested for 1 micro-liter saliva co-deposited on the same PTFE strip with varying amount of THC (10-100 pg). Saliva was used to mimic the biofluid condensed from exhaled breath, a simple way to test the potential interference on sensor performance. The sensor testing was performed with the same procedure using the same prototype detector equipped with front-end heater as described in Example 4 The sensor molecule used was also the same as used in Example 4, shown as Structure XVIII

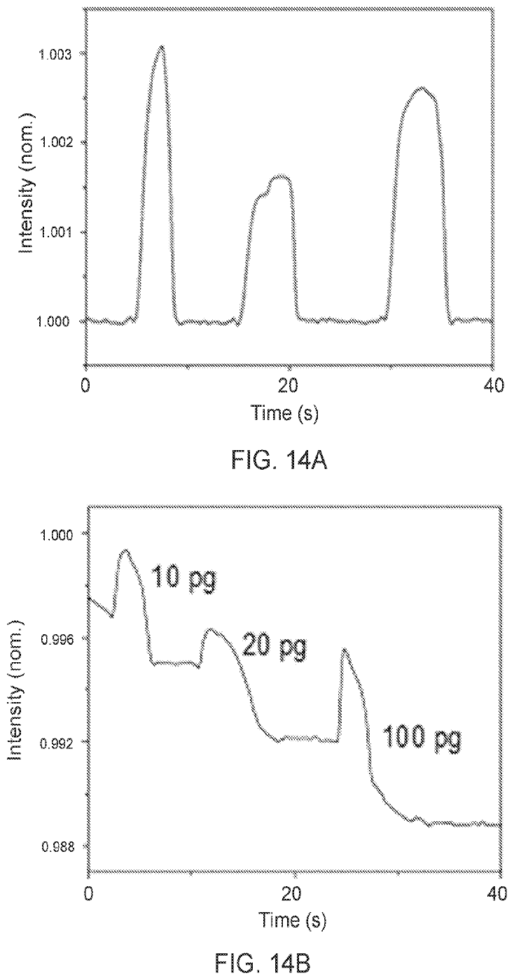

FIG. 14A shows sensor results using 1 micro-liter saliva deposited on the PTFE testing strip, and measured with the prototype detector equipped with frontend heater using the sensor molecule above. As can be seen, a quick reversible response observed with time span of ˜5 seconds.

FIG. 14B shows sensor results using 1 micro-liter saliva co-deposited with varying amount of THC (10, 20, 100 pg) on the PTFE testing strip, and measured with the prototype detector equipped with frontend heater using the sensor molecule above. The irreversible response (emission quenching) to THC can be clearly distinguished from the quick reversible response caused by the saliva (mostly due to the moisture). The response magnitude (relative decrease in emission intensity) is consistent with the results shown in Example 4, which was obtained from the testing with pure THC sample. Thus, THC can be reliably detected when mixed with biofluid like saliva. Accordingly, detecting THC from a real breath sample condensed on a substrate can also be performed.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

For example, various features and elements of the above-described invention can be used alone or in various combinations where elements descried in one example can be utilized in other described examples. More specifically, the invention can be generally described by the following clauses:

Clause 1. A fluorescent sensor compound for detecting marijuana, comprising an oligomer having a general structure of Y-[D]ₙ-A-Z wherein D is an electron donor group according to Formula (I):

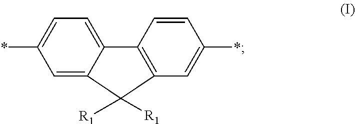

wherein A is an electron acceptor group according to Formula (11):

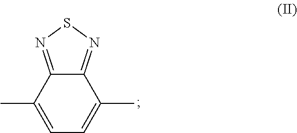

wherein Y is R₃, R₃-A, or R₃-D-A; and
wherein Z is R₃ or [D]ₘ-R₃;
wherein n is an integer from 1 to 3, wherein m is an integer from 1 to 2, wherein R₁ is an alkyl chain, wherein R₁ optionally includes different alkyl chains in different respective D groups, wherein R₃ is an alkoxyphenyl end group.

Clause 2. The compound of any Clause, wherein the alkyl chains are linear alkyl chains having from 1 to 8 carbon atoms.

Clause 3. The compound of any Clause, wherein the compound has a structure selected from the group consisting of Formula (III), Formula (IV), Formula (V), and Formula (VI):

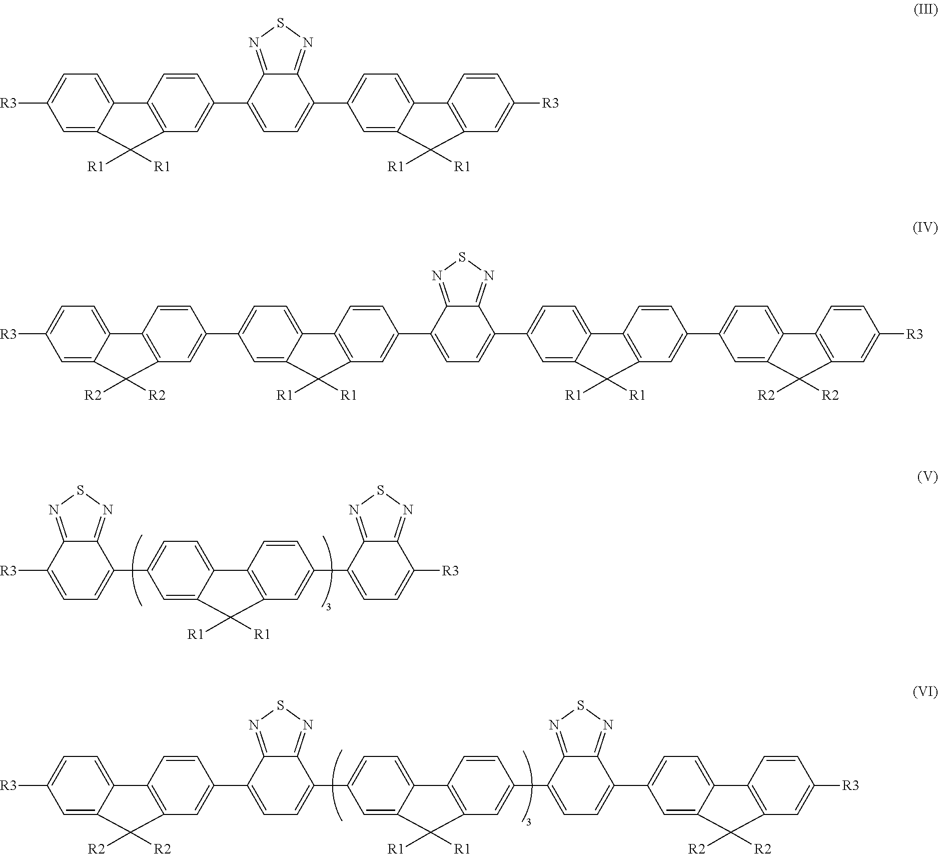

wherein R₁ and R₂ are independently alkyl groups selected from the group consisting of —CH₃, —C₂H₅, —C₃H₇, —C₄H₉, —C₅H₁₁, C₆H₁₃, C₇H₁₅, and C₈H₁₇; and wherein R₃ is an alkoxyphenyl group selected from the group consisting of Formula (VII), Formula (VIII), Formula (IX), Formula (X), Formula (XI), Formula (XII), Formula (XIII), Formula (XIV), Formula (XV), and Formula (XVI):

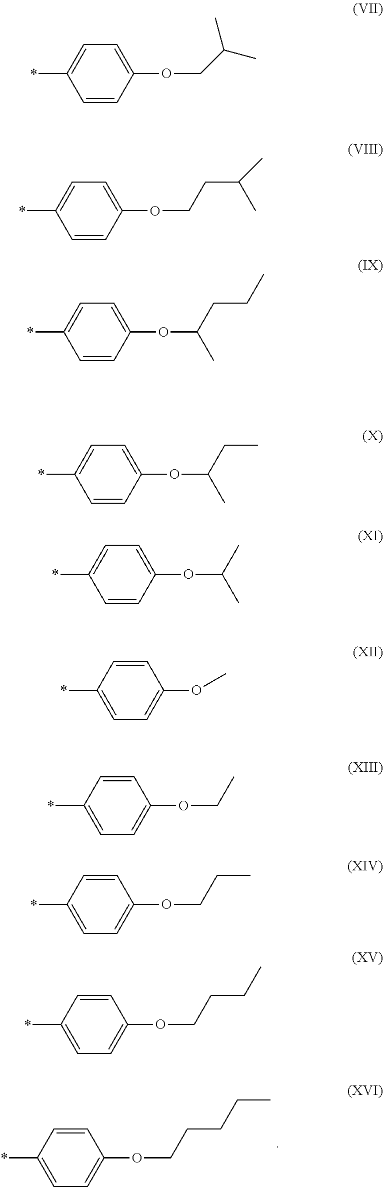

Clause 4. The compound of Clause 3, wherein the compound has the structure of Formula (V)

Clause 5. The compound of Clause 4, wherein R₁ is C₆H₁₃.

Clause 6 The compound of Clause 5, wherein R₃ has the structure of Formula (X).

Clause 7. A porous net structure of nanofibrils assembled from molecules of the compound of any Clause.

Clause 8. A fluorescent sensor for detecting marijuana, comprising:
a fluorescent sensor compound comprising an oligomer having a general structure of Y-[D]ₙ-A-Z wherein D is an electron donor group according to Formula (I):

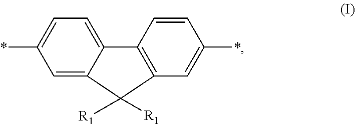

wherein A is an electron acceptor group according to Formula (II):

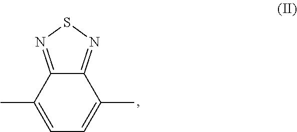

wherein Y is R₃, R₃-A, or R₃-D-A,
wherein Z is R₃ or [D]ₘ-R₃,
wherein n is an integer from 1 to 3, wherein m is an integer from 1 to 2, wherein Rr is an alkyl chain, wherein R₁ optionally includes different alkyl chains in different respective D groups, wherein R₃ is an alkoxyphenyl end group; and
a fluorescence detector oriented to detect a fluorescence response from the fluorescent sensor compound.

Clause 9. The sensor of any Clause, wherein the fluorescent sensor compound is present as a film having a thickness from about 100 nanometers to about 100 micrometers.

Clause 10. The sensor of any Clause, wherein the fluorescent sensor compound is present as a porous net structure of nanofibrils assembled from molecules of the fluorescent sensor compound.

Clause 11. The sensor of any Clause, wherein the sensor further comprises a housing having an inlet for exhaled breath.

Clause 12. The sensor of any Clause, wherein the housing directs the exhaled breath to the fluorescent sensor compound to detect marijuana in the exhaled breath directly.

Clause 13. The sensor of any Clause, further comprising a condensing surface, wherein the housing directs the exhaled breath to the condensing surface to form a breath condensate on the condensing surface, and wherein the sensor further comprises a heater to heat the breath condensate.

Clause 14. The sensor of any Clause, wherein the fluorescent sensor compound has a structure selected from the group consisting of Formula (III), Formula (IV), Formula (V), and Formula (VI):

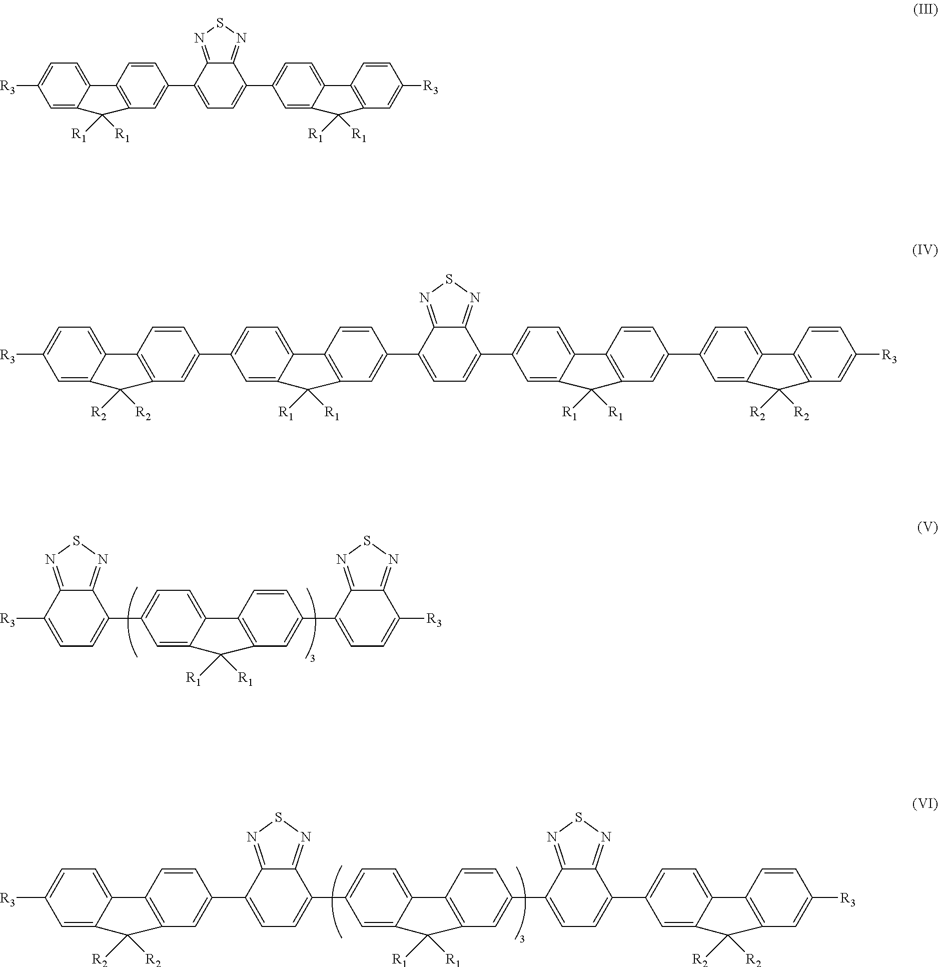

wherein R₁ and R₂ are independently alkyl groups selected from the group consisting of —CH₃, —C₂H₅, —C₃H₇, —C₄H₉, —C₅H₁₁, C₆H₁₃, C₇H₁₅, and C₈H₁₇; and wherein Rz is an alkoxyphenyl group selected from the group consisting of Formula (VII), Formula (VIII), Formula (IX), Formula (X), Formula (XI), Formula (XII), Formula (XIII), Formula (XIV), Formula (XV), and Formula (XVI):

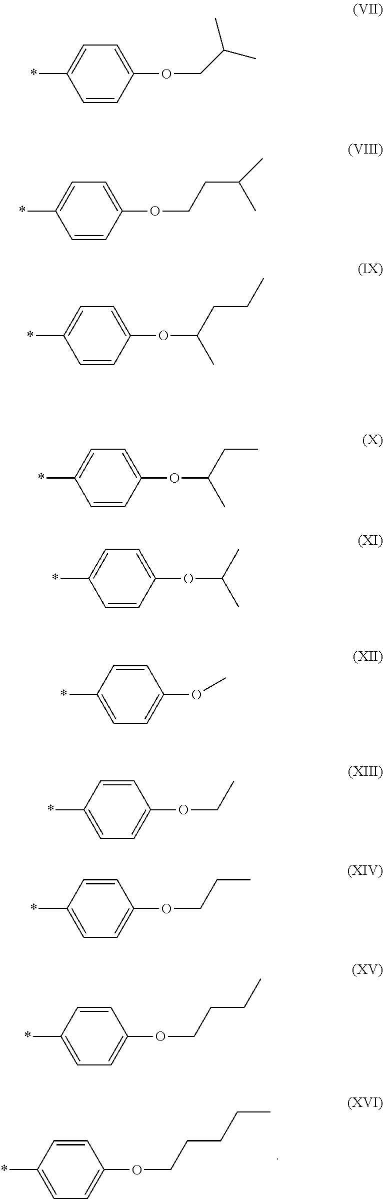

Clause 15. A method of detecting a marijuana breath marker, comprising:
exposing a fluorescent sensor compound to a breath sample, wherein the fluorescent sensor compound comprises an oligomer having a general structure of Y-[D]ₙ-A-Z wherein D is an electron donor group according to Formula (I):

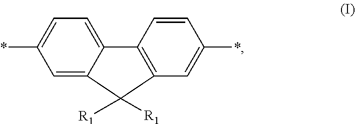

wherein A is an electron acceptor group according to Formula (II):

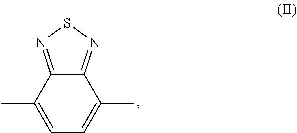

wherein Y is R₃, R₃-A, or R₁-D-A,
wherein Z is R₃ or [D]ₘ-R₃,
wherein n is an integer from 1 to 3, wherein m is an integer from 1 to 2, wherein R₁ is an alkyl chain, wherein R₁ optionally includes different alkyl chains in different respective D groups, wherein R₃ is an alkoxyphenyl end group;
correlating a fluorescence response of the fluorescent sensor compound to a target threshold; and
outputting an indicator corresponding to presence or absence of the marijuana breath marker.

Clause 16. The method of any Clause, wherein the marijuana breath marker is THC, cannabidiol, or a combination thereof.

Clause 17. The method of any Clause, wherein the marijuana breath marker is in exhaled breath at a concentration from 0.0008 ppbv to 0.2 ppbv.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A fluorescent sensor compound for detecting marijuana, comprising an oligomer having a general structure of Y-[D]ₙ-A-Z wherein D is an electron donor group according to Formula (I): 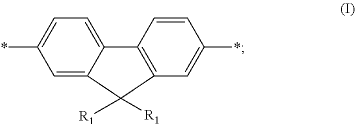
wherein A is an electron acceptor group according to Formula (II): 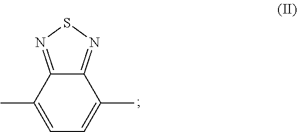
wherein Y is R₃, R₃-A, or R₃-D-A; and
wherein Z is R₃ or [D]ₘ-R₃;
wherein n is an integer from 1 to 3, wherein m is an integer from 1 to 2, wherein R₁ is an alkyl chain, wherein R₁ optionally includes different alkyl chains in different respective D groups, wherein R₃ is an alkoxyphenyl end group.

2. The compound of claim 1, wherein the alkyl chains are linear alkyl chains having from 1 to 8 carbon atoms.

3. The compound of claim 1, wherein the compound has a structure selected from the group consisting of Formula (III), Formula (IV), Formula (V), and Formula (VI): 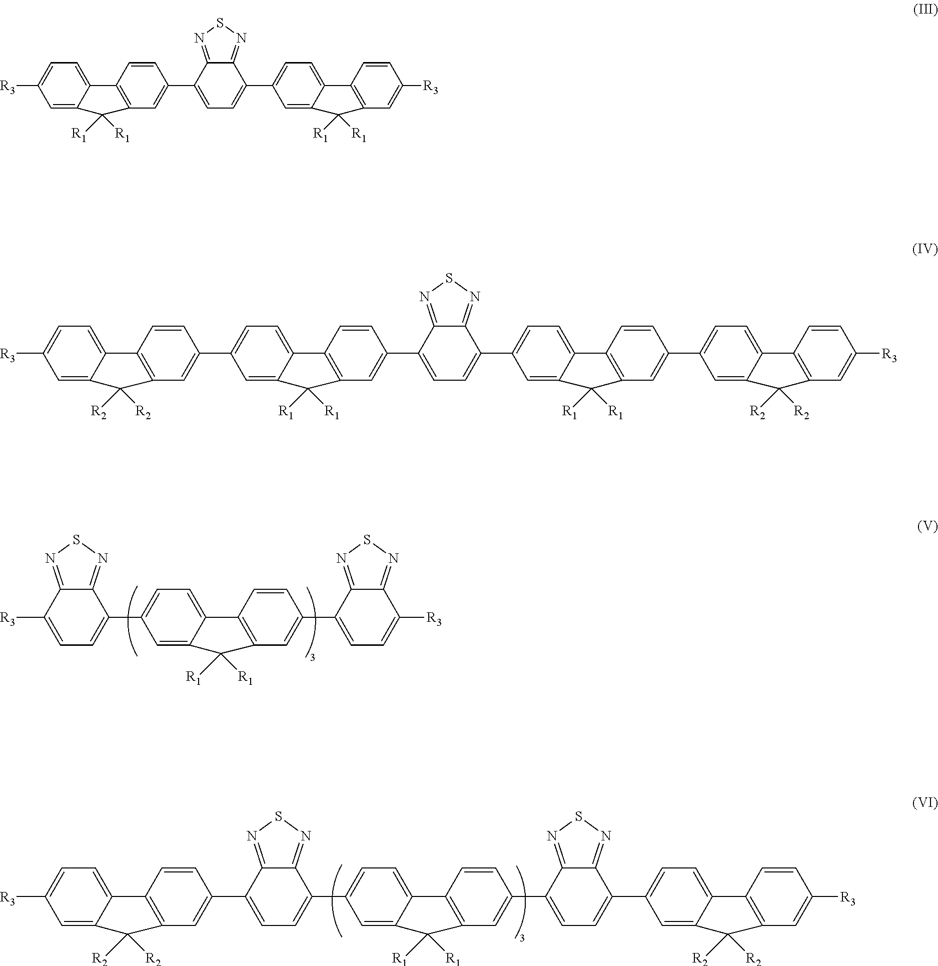
wherein R₁ and R₂ are independently alkyl groups selected from the group consisting of —CH₃, —C₂H₅, —C₃H₇, —C₄H₉, —C₅H₁₁, C₆H₁₃, C₇H₁₅, and C₈H₁₇; and wherein R₃ is an alkoxyphenyl group selected from the group consisting of Formula (VII), Formula (VIII), Formula (IX), Formula (X), Formula (XI), Formula (XII), Formula (XIII), Formula (XIV), Formula (XV), and Formula (XVI):

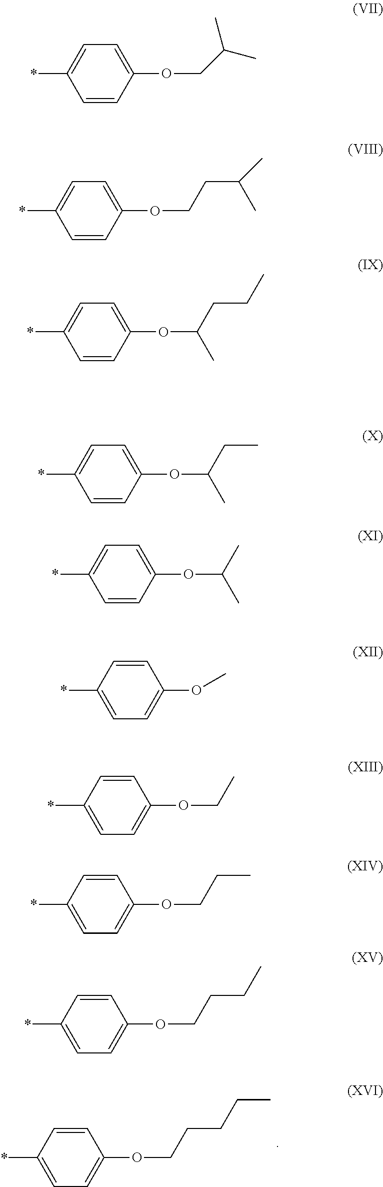

4. The compound of claim 3, wherein the compound has the structure of Formula (V).

5. The compound of claim 4, wherein R₁ is C₆H₁₃.

6. The compound of claim 5, wherein R₃ has the structure of Formula (X).

7. A porous net structure of nanofibrils assembled from molecules of the compound of claim 1.

8. A fluorescent sensor for detecting marijuana, comprising:
a fluorescent sensor compound comprising an oligomer having a general structure of Y-[D]ₙ-A-Z wherein D is an electron donor group according to Formula (I): 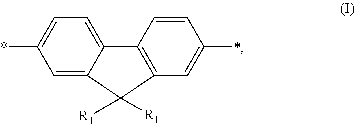
wherein A is an electron acceptor group according to Formula (II): 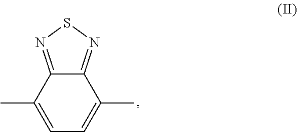
wherein Y is R₃, R₃-A, or R₃-D-A,
wherein Z is R₃ or [D]ₘ-R₃,
wherein n is an integer from 1 to 3, wherein m is an integer from 1 to 2, wherein R₁ is an alkyl chain, wherein R₁ optionally includes different alkyl chains in different respective D groups, wherein R₃ is an alkoxyphenyl end group; and
a fluorescence detector oriented to detect a fluorescence response from the fluorescent sensor compound.

9. The sensor of claim 8, wherein the fluorescent sensor compound is present as a film having a thickness from about 100 nanometers to about 100 micrometers.

10. The sensor of claim 8, wherein the fluorescent sensor compound is present as a porous net structure of nanofibrils assembled from molecules of the fluorescent sensor compound.

11. The sensor of claim 8, wherein the sensor further comprises a housing having an inlet for exhaled breath.

12. The sensor of claim 11, wherein the housing directs the exhaled breath to the fluorescent sensor compound to detect marijuana in the exhaled breath directly.

13. The sensor of claim 11, further comprising a condensing surface, wherein the housing directs the exhaled breath to the condensing surface to form a breath condensate on the condensing surface, and wherein the sensor further comprises a heater to heat the breath condensate.

14. The sensor of claim 8, wherein the fluorescent sensor compound has a structure selected from the group consisting of Formula (III), Formula (IV), Formula (V), and Formula (VI): 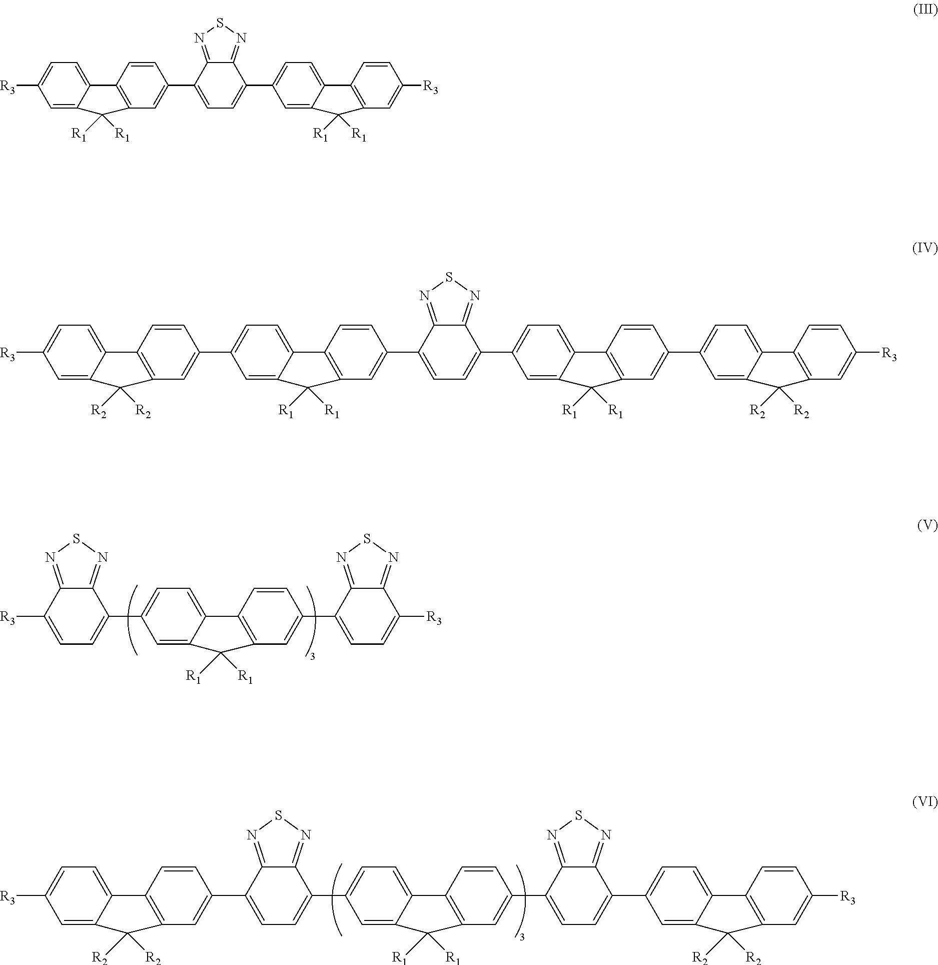
wherein R₁ and R₂ are independently alkyl groups selected from the group consisting of —CH₃, —C₂H₅, —C₃H₇, —C₄H₉, —C₅H₁₁, C₆H₁₃, C₇H₁₅, and C₈H₁₇; and wherein R₃ is an alkoxyphenyl group selected from the group consisting of Formula (VII), Formula (VIII), Formula (IX), Formula (X), Formula (XI), Formula (XII), Formula (XIII), Formula (XIV), Formula (XV), and Formula (XVI):

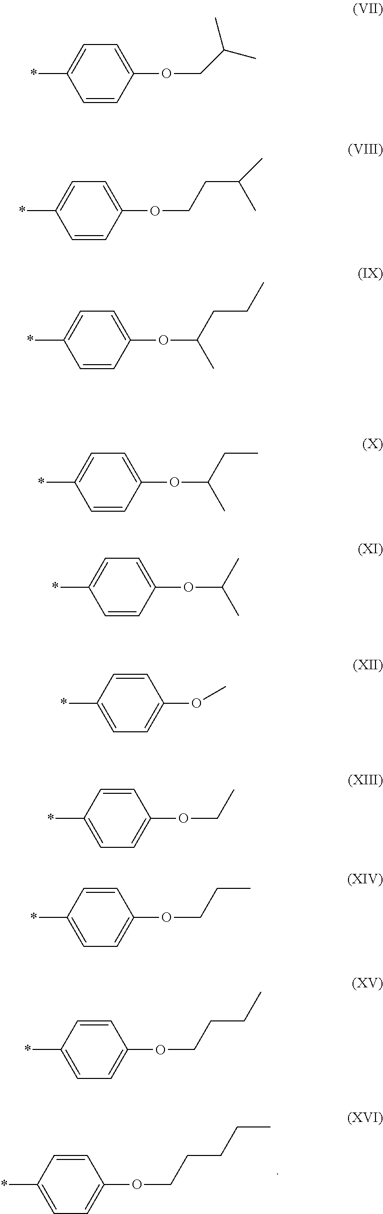

15. A method of detecting a marijuana breath marker, comprising:
exposing a fluorescent sensor compound to a breath sample, wherein the fluorescent sensor compound comprises an oligomer having a general structure of Y-[D]ₙ-A-Z wherein D is an electron donor group according to Formula (I): 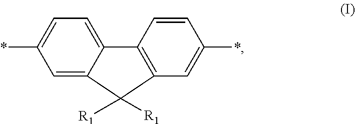
wherein A is an electron acceptor group according to Formula (II): 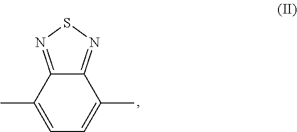
wherein Y is R₃, R₃-A, or R₃-D-A,
wherein Z is R₃ or [D]ₘ-R₃,
wherein n is an integer from 1 to 3, wherein m is an integer from 1 to 2, wherein R₁ is an alkyl chain, wherein R₁ optionally includes different alkyl chains in different respective D groups, wherein R₃ is an alkoxyphenyl end group;
correlating a fluorescence response of the fluorescent sensor compound to a target threshold; and
outputting an indicator corresponding to presence or absence of the marijuana breath marker.

16. The method of claim 15, wherein the marijuana breath marker is THC, cannabidiol, or a combination thereof.

17. The method of claim 15, wherein the marijuana breath marker is in exhaled breath at a concentration from 0.0008 ppbv to 0.2 ppbv.
